(19) United States
(12) Patent Application Publication
(10) Pub. No.: US 2017/0267998 A1
(43) Pub. Date: Sep. 21, 2017

(54) METHODS OF SYNTHESIZING POLYNUCLEOTIDES

(71) Applicant: AxioMx, Inc., Branford, CT (US)

(72) Inventor: Michael WEINER, Guilford, CT (US)

(21) Appl. No.: 15/532,452

(22) PCT Filed: Dec. 3, 2015
(86) PCT No.: PCT/US15/63716

§ 371 Date: Jun. 1, 2017

Related U.S. Application Data
(60) Provisional application No. 62/087,440, filed on Dec. 4, 2014.

(51) Int. Cl. C12N 15/10

(52) U.S. Cl. CPC C12N 15/1072; C12N 15/1031

(57) ABSTRACT

The present invention is directed to compositions and methods for producing one or more polynucleotides from smaller oligonucleotide segments within an emulsion. In methods of the present invention, a support having one or more capture oligo-nucleotides is contacted with two or more corresponding tile oligonucleotides. Upon hybridization of the tile oligonucleotides to the capture oligonucleotides, a capture complex is formed. This capture complex is emulsified, optionally with reaction reagents or other additives. The emulsion is then incubated at a temperature regimen sufficient for an adjoining extension reaction to occur, such that a polynucleotide may be formed from the tile oligonucleotides that hybridized to a particular support. A particular advantage of this method is that many different polynucleotides may be produced in parallel with surprising efficiency.

### BACKGROUND OF THE INVENTION

Current methods for polynucleotide synthesis may involve splicing together or otherwise assembling multiple oligonucleotides into a single nucleic acid molecule. These oligonucleotides may be approximately 60 to 100 nucleotides in length. In some protocols, numerous oligonucleotides are spliced together or otherwise assembled in a single bulk reaction to generate a polynucleotide. The complexity of the reaction increases, for instance, when the number or diversity of oligonucleotides in the reaction increases, the number or diversity of polynucleotides to be synthesized in the reaction increases, or the total length or diversity of the polynucleotides to be synthesized increases. However, as the complexity of the reaction increases, the likelihood of aberrant hybridization events between oligonucleotides, partially assembled polynucleotides, and/or assembled polynucleotides may also increase. Efforts to control this effect through systematic oligonucleotide design have proven largely insufficient and may undesirably affect codon usage. High error rates in polynucleotide synthesis can also be problematic in certain instances. As a result of at least these factors, existing methods of polynucleotide synthesis are not generally scalable or amenable to the parallel synthesis of a plurality of polynucleotides. Thus, there exists a need for scalable methods of polynucleotide synthesis.

### SUMMARY OF THE INVENTION

The present invention includes methods and compositions for producing polynucleotides, e.g., homologous variants. The present invention includes methods of generating a polynucleotide that may include the steps of providing a support and two or more tile oligonucleotides, such that the two or more tile oligonucleotides include overlapping, complementary segments of the polynucleotide, and the support has one or more capture oligonucleotides, such that a segment of each of the tile oligonucleotides may be complementary to at least one of the capture oligonucleotides; contacting the support with the tile oligonucleotides, such that the tile oligonucleotides hybridize to the capture oligonucleotides, thereby forming a capture complex; emulsifying the capture complex in an emulsion medium (e.g., a water-in-oil emulsion), the emulsion medium further including reaction reagents sufficient to carry out an adjoining extension reaction, such that the emulsion medium forms an emulsion droplet including the capture complex and the reaction reagents; and incubating the emulsion droplet at a temperature regimen that allows adjoining extension of the two or more tile oligonucleotides, thereby generating the polynucleotide. In such a method, each of the tile oligonucleotides may include an identifying sequence. In some embodiments, all of the tile oligonucleotides may include the same identifying sequence. In other embodiments, the tile oligonucleotides may include a plurality of distinct identifying sequences. In such embodiments, one of the tile oligonucleotides may be a base oligonucleotide that includes a first identifying sequence distinct from that of the remaining tile oligonucleotides.

In any of the above methods, one or more of the tile oligonucleotides may be provided as double-stranded tile oligonucleotides. In certain embodiments, one or more of the double-stranded tile oligonucleotides may be prepared from one or more single-stranded template oligonucleotides prior to providing the tile oligonucleotides.

In some methods of the present invention, a polynucleotide may be generated by a method that includes the steps of: synthesizing two or more double-stranded tile oligonucleotides from one or more single-stranded template oligonucleotides by providing one or more primers capable of hybridizing to one or more of the template oligonucleotides at one or more priming sequences, hybridizing the primers to the priming sequences, and extending the primers to produce one or more double-stranded tile oligonucleotides including a template strand and a newly synthesized strand; providing a support and two or more of the tile oligonucleotides, such that the two or more tile oligonucleotides may be overlapping, complementary segments of the polynucleotide, and the support has one or more capture oligonucleotides, such that a segment of each of the tile oligonucleotides may be complementary to at least one of the capture oligonucleotides; contacting the support with the tile oligonucleotides, such that the tile oligonucleotides hybridize to the capture oligonucleotides, thereby forming a capture complex, and emulsifying the capture complex in an emulsion medium, the emulsion medium further including reaction reagents sufficient to carry out an adjoining extension reaction, such that the emulsion medium forms an emulsion droplet including the capture complex and the reaction reagents; and incubating the emulsion droplet at a temperature regimen that allows adjoining extension of the two or more tile oligonucleotides, thereby generating the polynucleotide. In some embodiments of this method, two or more of the template oligonucleotides may include identical priming sequences. In other embodiments, the template oligonucleotides may include a plurality of priming sequences.

In certain embodiments, the synthesizing may be by solid state synthesis from template oligonucleotides affixed at the 5′ terminus to a solid state synthesis structure, the synthesizing step further including providing a cleavage reagent after the extending and incubating the solid state synthesis structure with the cleavage reagent and the template oligonucleotides further including one or more cleavage sites for the cleavage reagent positioned 5′ of the segment of the template oligonucleotide corresponding to the polynucleotide, thereby producing one or more tile oligonucleotides each including a template strand and a newly synthesized strand. In other embodiments, the synthesizing may be by solid state synthesis from template oligonucleotides affixed at the 3′ terminus to a solid state synthesis structure, the synthesizing step further including providing a cleavage reagent after the extending and incubating the solid state synthesis structure with the cleavage reagent and the template oligonucleotides further including one or more cleavage sites for the cleavage reagent positioned 3′ of the segment of the template oligonucleotide corresponding to the polynucleotide, thereby producing one or more tile oligonucleotides each including a template strand and a newly synthesized strand.

In certain embodiments, the template oligonucleotides may be initially affixed at the 5′ terminus to a solid state synthesis structure, the synthesizing step further including providing a cleavage reagent prior to the extending and incubating the solid state synthesis structure with the cleavage reagent, such that the template oligonucleotides further include one or more cleavage sites for the cleavage reagent positioned 5′ of the segment of the template oligonucleotide corresponding to the polynucleotide, thereby producing one or more free single-stranded template oligonucleotides prior to the extending. In other embodiments, the template oligonucleotides may be initially affixed at the 3′ terminus to a solid state synthesis structure, the synthesizing step further including providing a cleavage reagent prior to the extending and incubating the solid state synthesis structure with the cleavage reagent, such that the template oligonucleotides further include one or more cleavage sites for the cleavage reagent positioned 3′ of the segment of the template oligonucleotide corresponding to the polynucleotide, thereby producing one or more free single-stranded template oligonucleotides prior to the extending.

In some embodiments, the primers capable of hybridizing to one or more of the template oligonucleotides may be primers for a strand-displacing polymerase and the extending may be by a single round of strand-displacing extension. In some embodiments, the extending step may be performed in an emulsion including the single-stranded template oligonucleotides, primers, and polymerase. In certain embodiments, the method further includes breaking the emulsion to produce a solution including one or more double-stranded tile oligonucleotides. In particular embodiments, each of the template oligonucleotides includes an identifying sequence, whereby each double-stranded tile oligonucleotide includes the identifying sequence present in the template oligonucleotide from which it was synthesized.

In certain embodiments, the priming sequence may be 5′ of the identifying sequence, thereby generating a tile oligonucleotide in which the 3′ end of the template strand includes a single-stranded 3′ overhang that extends beyond the 5′ end of the newly synthesized strand and includes the identifying sequence. In some embodiments, one or more of the primers includes a 5′ phosphate and hybridizes specifically to a template oligonucleotide encoding a base oligonucleotide, whereby the synthesis results in the base oligonucleotide including a newly synthesized strand including a 5′ phosphate, and the step of contacting the support to the tile oligonucleotides further includes contacting ligase to the support and incubating the ligase, support, and tile oligonucleotides together prior to the emulsification, whereby one or more of the newly synthesized strands including a 5′ phosphate may be covalently joined by the activity of the ligase to a capture oligonucleotide of the capture complex.

In any of the above embodiments, one or more of the tile oligonucleotides hybridized to the capture oligonucleotides in the capture complex may further include cleavage sites positioned such that cleavage at one or more of the cleavage sites liberates from the capture complex a portion of one or more of the tile oligonucleotides including the segment corresponding to the polynucleotide, the method further including contacting the capture complex with one or more cleavage reagents. In some embodiments, all of the tile oligonucleotides may include a cleavage site. In certain embodiments, one or more of the tile oligonucleotides may be base oligonucleotides and all of the tile oligonucleotides except the base oligonucleotides include a cleavage site.

In particular embodiments, one strand of one or more of the double-stranded tile oligonucleotides may be protected and the second strand may be non-protected, the method further including the step of selectively degrading the non-protected strand over the protected strand prior to the incubation at a temperature regimen that allows adjoining extension, thereby producing a single-stranded tile oligonucleotide. In such embodiments, the non-protected strand may include two or fewer 5′ phosphorothioate groups, the protected strand may include three or more 5′ phosphorothioate groups, and the degrading may include incubating the tile oligonucleotides with an enzyme capable of selectively degrading a strand having two or fewer 5′ phosphorothioate groups over a strand having three or more 5′ phosphorothioate groups. In these embodiments, the enzyme capable of selective degradation may be T7 exonuclease or lambda exonuclease. In some embodiments, the non-protected strand may include methylated nucleobases, the protected strand may lack methylated nucleobases, and the degrading may include incubating the tile oligonucleotides with an enzyme capable of selectively degrading a methylated strand over a strand that may be not methylated. In these embodiments, the non-protected strand may include methylated adenine nucleobases and the enzyme capable of selective degradation may be DpnI. In some embodiments, the non-protected strand may include methylated cytosine nucleobases and the enzyme capable of selective degradation may be mcrBC. In these embodiments, the non-protected strand may lack methylated nucleobases, the protected strand may include methylated nucleobases, and the degrading may include incubating the tile oligonucleotides with an enzyme capable of selectively degrading a non-methylated strand over a methylated strand. In such embodiments, the protected strand may include methylated cytosine or guanine nucleobases and the enzyme that selectively degrades non-methylated nucleic acids may be Sau3AI. In other embodiments, the non-protected strand includes deoxyuracil and the enzyme capable of selective degradation is dut. In some embodiments, the non-protected strand may include uracil, the protected strand may lack uracil, and the degrading may include incubating the tile oligonucleotides with an enzyme capable of selectively degrading a uracilated strand over a strand that may not be uracilated, thereby producing a single-stranded tile oligonucleotide. In such embodiments, the enzyme that selectively degrades uracilated nucleic acids may be a uracil-DNA glycosylase. In any method in which one strand of one or more of the double-stranded tile oligonucleotides may be protected and the second strand may be non-protected the protected strand may be the template strand. In some embodiments, the step of selectively degrading the template strand occurs after the contacting of the capture complex with the cleavage reagent. Alternatively, the protected strand may be the newly synthesized strand. In such embodiments, that the step of selectively degrading the newly synthesized strand may occur prior to the contacting of the capture complex with the cleavage reagent, prior to the emulsifying, or after the contacting of the capture complex with the cleavage reagent. In particular embodiments, the step of selectively degrading the newly synthesized strand occurs prior to the emulsifying. In certain embodiments, the step of selectively degrading the newly synthesized strand occurs after the contacting of the capture complex with the cleavage reagent.

In any of the above embodiments, each of the tile oligonucleotides may be 20 bp to 2 kb in length. In any of the above embodiments, the support may include capture nucleotides synthesized to hybridize to the identifying sequences of tile oligonucleotides corresponding to a single polynucleotide. In any of the above embodiments, the support may include capture nucleotides synthesized to hybridize to the identifying sequences of tile oligonucleotides corresponding to two to ten distinct polynucleotides. The support may include 1 to 1,000 distinct capture oligonucleotides, preferably 2 to 50 distinct capture oligonucleotides. In any of the above embodiments, the reaction reagents may be sufficient to carry out a SO-PCR reaction and the adjoining extension reaction may be SO-PCR. Alternatively, the reaction reagents may be sufficient to carry out a Gibson Assembly reaction such that the adjoining extension reaction may be a Gibson Assembly reaction. In any of the above embodiments, the extension reaction may include a temperature regimen sufficient to denature and subsequently reanneal overlapping, complementary sequences of the tile oligonucleotides.

In any of the above embodiments, the emulsifying may include a plurality of supports and the resulting emulsion may include a plurality of droplets. Each droplet of the emulsion may contain 0-10 supports, such as, on average, 0-2 supports, or, on average, 1 support.

Any of the above methods may further include the step of breaking the emulsion, thereby producing a solution including one or more supports and one or more polynucleotides. Such embodiments may include the still further step of purifying the polynucleotides or purifying the supports. In embodiments in which the supports may be purified, the supports may include one or more detectable labels, and the method may further include, after the step of breaking the emulsion, the step of sorting the supports according to the one or more detectable labels. In certain embodiments, the method further includes incubating the polynucleotides with ligase after breaking the emulsion, thereby forming covalent bonds at nicks in the polynucleotide. In any of the above embodiments, the polynucleotide may be 50 bp to 20 kb in length, preferably 100 bp to 10 kb.

In any of the above embodiments, the emulsion may include a plurality of supports corresponding to distinct polynucleotides and the incubation of the emulsion at a temperature regimen that allows adjoining extension may result in the generation of a plurality of distinct polynucleotides, such that the method may further include generating a plurality of distinct polynucleotides, each polynucleotide being generated within an emulsion droplet containing the corresponding support. Any of the above embodiments may further include the step of amplifying the polynucleotides. In particular embodiments, two or more polynucleotides present in an emulsion include one or more variable priming sequences having one or more distinct permutations positioned 3′ of a sequence of interest on one or both strands of each of the polynucleotides, the method further including the steps of: providing one or more permutation-specific primers and amplification reaction reagents; contacting the polynucleotides with the permutation-specific primers in the presence of the amplification reaction reagents; and incubating the polynucleotides together with the permutation-specific primers in the presence of the amplification reaction reagents at a temperature regimen that allows amplification, thereby selectively amplifying polynucleotides having a particular permutation of a variable priming sequence. In other embodiments, two or more polynucleotides present in an emulsion include one or more variable priming sequences having one or more distinct permutations and one or more non-variable priming sequences positioned 3′ of a sequence of interest on one or both strands of the polynucleotide, the method further including the steps of: providing one or more primers capable of hybridizing to the non-variable priming sequences and a first set of amplification reaction reagents; contacting the polynucleotides to the non-variable priming sequence primers in the presence of the first set of amplification reaction reagents; incubating the polynucleotides together with the non-variable priming sequence primers and the first set of amplification reaction reagents at a temperature regimen that allows amplification, thereby producing amplicons of polynucleotides having the non-variable priming sequence; removing and sequencing a subset of amplicons to identify polynucleotide sequences having a particular sequence and one or more associated variable priming sequence permutations; providing one or more permutation-specific primers and a second set of amplification reaction reagents; contacting the remaining polynucleotides and/or amplicons with the permutation-specific primers in the presence of the second set of amplification reaction reagents; and incubating the remaining polynucleotides and/or amplicons together with the permutation-specific primers in the presence of the amplification reaction reagents at a temperature regimen that allows amplification, thereby selectively amplifying polynucleotide sequences having a particular permutation of a variable priming sequence.

In any of the above embodiments, the support may be a bead, chip, tube, or well. In some embodiments, the bead may be a magnetic bead. In certain embodiments, the bead may be labeled. In such embodiments, the label may be a colored label or a fluorescent label. In particular embodiments, the fluorescent label may be a dye or fluorescent protein. In any of the above embodiments, the emulsifying step may include emulsifying the capture complex in a water-in-oil emulsion. In further embodiments, the water-in-oil emulsion may be a water-in-perfluorocarbon oil emulsion. In certain embodiments, the emulsifying step may include the use of a mechanical device to emulsify the capture complex in the emulsion medium. In such embodiments, the mechanical device may be a stirrer, homogenizer, colloid mill, ultrasound, membrane emulsification device, or vortex. In any of the above embodiments, the emulsion medium may further include a recA protein, recA peptide, or a crowding agent. In particular embodiments, the crowding agent may be polyethylene glycol or hexamine cobalt chloride. In some embodiments, the polynucleotide may encode one or more complementarity determining regions. In certain embodiments, the polynucleotide may encode CDR-L1, CDR-L2, CDR-L3, CDR-H1, CDR-H2, and/or CDR-H3.

Certain methods of the present invention may be for the selective amplification of one or more synthesized polynucleotides and may include the steps of: providing a pool of polynucleotides, each polynucleotide including one or more variable priming sequences on one or both strands of the polynucleotide 3′ of a sequence to be amplified, one or more permutation-specific primers, and amplification reaction reagents, such that the polynucleotides present in the pool include one or more distinct permutations of one or more of the variable priming sequences; contacting the pool of synthesized polynucleotides with the permutation-specific primers in the presence of the amplification reaction reagents; and incubating the polynucleotides together with the permutation-specific primers in the presence of the amplification reaction reagents at a temperature regimen that allows amplification, thereby selectively amplifying polynucleotides having a particular distinct permutation of a variable priming sequence. In particular methods, two or more of the polynucleotides may include distinct permutations of one or more of the variable priming sequences. The variable priming sequence may include two or more variable nucleotide positions, two to six variable nucleotide positions, or four variable nucleotide positions. The variable priming sequence may consist of eight to thirty nucleotides. The non-variable nucleotide positions of the variable priming sequences may be constant positions. In embodiments including one or more variable priming sequences, one or more polynucleotides that are otherwise identical may include distinct variable priming sequence permutations. In certain embodiments, one or more of the two or more polynucleotides that are otherwise identical, e.g., homologous, may encode a variant of the sequence of interest. In particular embodiments, permutation-specific primers may hybridize selectively to the permutation present in a polynucleotide encoding the sequence of interest or to the permutation present in a polynucleotide encoding a variant of the sequence of interest. A variable nucleotide position may include an adenine, guanine, cytosine, thymine, or uracil nucleotide. In some embodiments, a variable nucleotide position may include a nucleotide other than adenine, guanine, cytosine, thymine, or uracil. The variable nucleotide position may include a synthetic nucleotide. The variable nucleotide positions present in a variable priming sequence may be contiguous or may not be contiguous. In some embodiments, an amplicon of a polynucleotide having a distinct variable priming site permutation may be sequenced and the polynucleotide may be consequently selected for the selective amplification.

The invention includes a complex including a support and one or more tile oligonucleotides, such that the support may include one or more capture oligonucleotides hybridized to the one or more tile oligonucleotides and the tile oligonucleotides may be complementary, overlapping segments of a polynucleotide.

The invention further includes a solution including two or more tile oligonucleotides and one or more supports, such that the two or more tile oligonucleotides may be complementary, overlapping segments of a polynucleotide and the supports may include two or more capture oligonucleotides capable of hybridizing to the tile oligonucleotides.

The invention additionally includes a method of performing an adjoining extension reaction in an emulsion, such that an emulsion droplet may include segments including sequences to be adjoined and reaction reagents sufficient to carry out an adjoining extension reaction may be incubated at a temperature regimen sufficient to perform the adjoining extension reaction, whereby the sequences to be adjoined produce a single polynucleotide. In certain embodiments, the reaction reagents may be sufficient to carry out a SO-PCR reaction and the adjoining extension may include SO-PCR or the reaction reagents may be sufficient to carry out a Gibson Assembly reaction and the adjoining extension may include a Gibson Assembly reaction.

### Definitions

“Support” means any structure (e.g., a solid structure) capable of directly or indirectly interacting with an oligonucleotide. As used herein, the support together with an associated oligonucleotide may be incorporated into a droplet of an emulsion. A support may be a bead (e.g., a Luminex bead), such as a fluorescent bead. The bead may be designed for use in molecular biology.

“Polynucleotide” means a nucleic acid having one or two strands. Two polynucleotides will be referred to as “distinct” if they differ by at least one nucleotide.

A “variant of a polynucleotide” means a sequence derived from the sequence of a reference polynucleotide that contains one or more sequence changes as compared to the reference polynucleotide. A variant of a polynucleotide may have 70%-99.9% identity with the polynucleotide, such as about 70%, 75%, 80%, 85%, 90%, 95%, 96%, 97%, 98%, 99%, 99.5%, or 99.9% identity. A variant may be substantially identical to the polynucleotide.

“Tile oligonucleotide” means a nucleic acid molecule that has at least one segment of one strand that is capable of hybridizing to a segment of one strand of a polynucleotide of interest. The segment of a tile oligonucleotide capable of hybridizing to a segment of one strand of a polynucleotide of interest, or a template for synthesizing such a segment, is a “hybridization segment.” A tile oligonucleotide may comprise additional nucleotides that are not capable of hybridizing to a polynucleotide. For instance, a tile oligonucleotide may additionally include one or more identifying sequences, priming sequences, or cleavage sequences. A tile oligonucleotide may be single-stranded, double stranded, or inclusive of single-stranded and double stranded regions contiguously linked by nucleic acid interactions. A tile oligonucleotide may be referred to as a “double-stranded tile oligonucleotide” if it comprises two complementary strands over any segment of its length. Thus, a double-stranded tile oligonucleotide may be a tile oligonucleotide that comprises both double- and single-stranded regions. A tile oligonucleotide will be said to “correspond” to a polynucleotide or segment thereof if the tile oligonucleotide includes a segment that are capable of hybridizing to that polynucleotide or segment thereof.

A plurality of tile oligonucleotides having segments capable of hybridizing to a single polynucleotide may be designed. These tile oligonucleotides corresponding to a single polynucleotide may be designed such that each tile oligonucleotide corresponds to a distinct segment of the polynucleotide. They may be designed such that at least one tile oligonucleotide contains a sequence that corresponds to each nucleotide position of the polynucleotide. Tile oligonucleotides corresponding to the entirety of a polynucleotide may be ordered along the length of the polynucleotide sequence. The segments to which the tile oligonucleotides correspond may overlap such that terminal nucleotides of one tile oligonucleotide are complementary to the terminal nucleotides of another tile oligonucleotide. “Overlap” will occur when the hybridization segments of two tile oligonucleotides correspond to opposite strands of a single segment of the same polynucleotide, such that 3′ terminal nucleobases of the hybridization segment of one tile oligonucleotide are capable of hybridizing to 5′ terminal nucleobases of the hybridization segment of the second. Two or more tile oligonucleotides collectively corresponding to the entirety of a polynucleotide may be synthesized such that their hybridization segments form a series of overlapping segments, the terminus of each complementary to the opposing terminus of the next. Two or more tile oligonucleotides having hybridization segments so arranged and directed to a single polynucleotide form a “set” of tile oligonucleotides. Two sets of tile oligonucleotides will be referred to as “distinct” if they are directed to distinct polynucleotides or if they are directed to the same polynucleotide but one set is synthesized to include a tile oligonucleotide that is distinct from any present in the other set.

“Identifying sequence” means a segment of a tile oligonucleotide that may be used to isolate or collect the tile oligonucleotide. In some instances, an identifying sequence will provide a means of differentiating, preferentially isolating, or preferentially collecting one tile oligonucleotide or set of tile oligonucleotides from another tile oligonucleotide or set of tile oligonucleotides. An identifying sequence may be a single-stranded segment of a tile oligonucleotide. To isolate or collect one or more tile oligonucleotides, a pool of tile oligonucleotides having identifying sequences may be contacted with a probe capable of specifically interacting with one or more particular identifying sequences. For instance, the probe may be a nucleic acid probe having a segment complementary to the identifying sequence. In certain instances, the probe is a barcode and the identifying sequence is complementary to the barcode (e.g., a complementary barcode or cBarcode). The probe may then be sorted or separated from other probes by a variety of mechanisms known in the art. In some examples, the probe may be bound to a support (e.g., a bead).

“Capture oligonucleotide” means a support-bound nucleic acid molecule having at least one segment of one strand that is capable of hybridizing to an identifying sequence of one or more tile oligonucleotides. A tile oligonucleotide may be single-stranded, double stranded, or inclusive of single-stranded and double stranded regions contiguously linked by nucleic acid interactions. A capture oligonucleotide will be said to “correspond” to a tile oligonucleotide if that tile oligonucleotide includes an identifying sequence to which the capture oligonucleotide is capable of hybridizing. A capture oligonucleotide will be said to “correspond” to a polynucleotide if that capture oligonucleotide is capable of hybridizing to the identifying sequence of a tile oligonucleotide that corresponds to that polynucleotide. Further, a support having one or more capture oligonucleotides corresponding to a tile oligonucleotide will be said to “correspond” to that tile oligonucleotide, as well as to the corresponding polynucleotide. In the context of a support having capture oligonucleotides, the term “support” may refer to the support itself or to the support and its capture oligonucleotides together.

“Capture complex” means a complex that is formed when a support having one or more capture oligonucleotides is contacted with one or more corresponding tile oligonucleotides. A capture complex constitutes a support having one or more capture oligonucleotides to which one or more corresponding tile oligonucleotides are hybridized. A support having one or more capture oligonucleotides becomes a capture complex upon hybridization to one or more corresponding tile oligonucleotides. Accordingly, the occupancy of capture oligonucleotides present on a capture complex may be 100%, less than 100%, less than 90%, less than 80%, less than 70%, less than 60%, less than 50%, less than 25%, less than 20%, less than 15%, less than 10%, less than 5%, or less than 1%. For instance, the occupancy of capture oligonucleotides present on a capture complex may be 100%, 90%, 80%, 70%, 60%, 50%, 40%, 30%, 20%, 10%, 5%, 1%, 0.5%, 0.1%, 0.01%, 0.001%, or 0.0001%.

“Base oligonucleotide” means a tile oligonucleotide that is a terminal oligonucleotide in a set of tile oligonucleotides (i.e., it corresponds to a terminal segment of a polynucleotide). The term base oligonucleotide further means that this nucleotide is engineered to remain joined to a capture complex under conditions sufficient to liberate other tile oligonucleotides. In some instances, a set of tile oligonucleotides will include one base oligonucleotide. In other instances, a set of tile oligonucleotides will include two or more base oligonucleotides. In still other instances, a set of tile oligonucleotides will include no base oligonucleotide.

“Degrade” means to alter a DNA molecule by cleaving, removing, or replacing nucleobases by treatment with an enzyme or chemical.

“Protected” means that a nucleic acid molecule is degraded more slowly as compared to a non-protected nucleic acid molecule under the same conditions. A protected nucleic acid molecule may be a DNA molecule that is not degraded at all. A protected nucleic acid molecule may be a nucleic acid molecule that includes modified nucleobases, nucleobases other than adenine, guanine, cytosine, and thymine, or inter-nucleotide bonds other than phosphodiester bonds, such that degradation of the nucleic acid molecule is inhibited as compared to the degradation of a non-protected DNA molecule. Alternatively, a protected DNA molecule may be a DNA molecule that is not modified in a manner capable of increasing the degradation of the DNA molecule as compared to the degradation of a DNA molecule that is so modified.

“Selective degradation” means that a provided enzyme degrades some DNA molecules more rapidly than others. For example, the enzyme may selectively degrade a non-protected strand over a protected strand. A non-protected strand may be selectively degraded at a rate, for example, of at least about 20-fold greater than the rate at which the protected strand is degraded. In some cases, the enzyme may not degrade the protected strand at all.

“Adjoining extension reaction” means a process by which two or more nucleic acids are hybridized and, optionally, enzymatically extended to form a polynucleotide that is greater in length than any one of the starting nucleic acids.

“Reaction reagents” means a set of reagents sufficient to practice a selected method of amplification or adjoining extension.

“Contiguous” tile oligonucleotides or hybridization segments means two or more overlapping hybridization segments corresponding to a single polynucleotide and joined by hybridization of the overlapping nucleotides. Typically, contiguous association involves two or more hybridization segments capable of hybridizing to a first strand of a polynucleotide joined by overlapping, complementary nucleotides and at least one hybridization segment capable of hybridizing to the opposing strand of the polynucleotide. A series of contiguously associated hybridization segments may form a polynucleotide or polynucleotide segment having both double-stranded and single-stranded regions: double-stranded regions formed where segments overlap and single-stranded regions that intercalate the regions of overlap. Alternatively, two hybridization segments capable of hybridizing to a given strand of a polynucleotide may be perfectly adjacent, such that no polynucleotide bases separate the 5′ terminus of one from the 3′ terminus of the next, resulting in a nick.

“Amplification” means any method of producing directly or indirectly, perfectly or imperfectly, one or more nucleic acid strands based upon the sequence content of a template nucleic acid molecule. The produced strand or strands may be identified as an “amplicon.” The template may be single stranded or double stranded. The template may be a polynucleotide or an amplicon. An amplicon may be produced from either strand of a double stranded template. An amplicon may include the same nucleobases as a template, complementary nucleobases, or nucleobases that vary from the template in sequence content or chemistry. In some embodiments, a DNA amplicon will be produced from a DNA template. In alternative embodiments a DNA template may be used to produce an RNA amplicon, for instance by an RNA polymerase. In still other examples, the amplicon will include modified or artificial nucleobases, which may or may not have been present in the template. Methods of amplification, for example PCR, are well known in the art.

### BRIEF DESCRIPTION OF THE DRAWINGS

FIG. 1 is a diagram showing a method of splice-overlap polymerase chain reaction (SO-PCR). In this illustration, initial PCR(s) generate overlapping gene segments that are then used as PCR templates in a subsequent PCR. This initial reaction utilizes internal primers synthesized to overlap and thereby generate overlapping, complementary ends on initial PCR products. The primers may introduce nucleotide substitutions, insertions or deletions for site-directed mutagenesis or gene splicing. In the present invention, this first PCR may be substituted with the synthesis of overlapping tile oligonucleotides. Following the first PCR (or the provision of overlapping oligonucleotides), a subsequent PCR is performed to produce a full-length product. As illustrated, overlapping strands of initial PCR products hybridize at 3′ segments and are extended to generate a full-length product that is amplified by primers flanking the product. Flanking primers can incorporate restriction enzyme sites for inserting the product into an expression vector for cloning purposes.

FIG. 2 is a diagram showing a generic method of Gibson Assembly. Gibson Assembly is commonly used for in vitro assembly. The reaction requires only input DNA with appropriate overlaps and an appropriate mix of three enzymes, as found in a Gibson Assembly Master Mix. The Gibson Assembly Master Mix includes three different enzymatic activities that perform in a single buffer: the exonuclease creates a single-stranded 3′ overhang that facilitates the annealing of fragments that share complementarity at one end; the polymerase fills in gaps within each annealed fragment; and the DNA ligase (e.g., T4 DNA ligase) seals nicks in the assembled DNA. As illustrated, DNA fragments are added to the master mix and incubated at 50° C. for 1 hour; the resulting assembly product is a fully sealed dsDNA suitable for a range of downstream applications. Intermediate steps are illustrated in the right-hand panel.

FIG. 3 is a diagram showing a generic method of Gibson Assembly. Gibson Assembly efficiently joins multiple overlapping DNA fragments in a single-tube isothermal reaction. Gibson assembly may be used to produce a construct for transformation into competent E. coli.

FIG. 4 is a diagram showing a method of emulsion-based gene synthesis. The illustrated method is amenable to parallel synthesis of many polynucleotides. Step A shows a template oligonucleotide affixed to a chip synthesis structure. The template oligonucleotide, which may be attached to the chip synthesis structure, e.g., at the 3′ end, includes a DNA sequence complementary to a nucleic acid barcode (the “complementary barcode” or “cBarcode”) and an adjacent universal priming site. The cBarcode may be encoded at the 5′ end of each microarray-synthesized strand. Type ‘A’ oligonucleotides are the only oligonucleotides including a 5′ phosphate at their terminus and have a separate universal primer unique to all ‘A’ oligonucleotides. Step B shows the hybridization of a universal primer to the universal priming site of the template oligonucleotide. As shown, the primer binds downstream of the cBarcode sequence, such that the cBarcode sequence remains single stranded. Polymerase and nucleotides comprising dATP, dGTP, dUTP, and 5-methyl-dCTP are further provided, and the hybridized primer extends toward the base of the chip. To place the barcode at the opposite end, an unnatural base (e.g., a phosphoramidite spacer) may be incorporated into the synthesized DNA. In step C, a cleavage agent is used to cleave the double-stranded product liberating the tile oligonucleotides from the chip. Steps B and C are shown as being performed on an array, but may alternatively be performed in an emulsion. In steps D and E, tile oligonucleotides are incubated with beads attached to the nucleic acid barcodes, resulting in hybridization of the tile oligonucleotide cBarcodes to the respective barcodes attached to the beads. In step F, the addition of ligase results in covalent attachment of the base oligonucleotide ‘A’ to a corresponding barcoded bead. In steps G-I, the beads are emulsified with a cleavage agent, and the methyl-cytosine restricting enzyme mcrBC is added to selectively degrade the newly synthesized, methylated tile oligonucleotide strands, which results in removal of the barcodes. Incubation at 95° C. denatures the tile oligonucleotides, and a subsequent annealing step allows overlapping hybridization segments to produce a nicked polynucleotide. The emulsion may then be broken, and the beads may be washed. As shown in step J, ligase may be added to seal the nicks. In one example, a PCR reaction (e.g., emulsion PCR) may be used to amplify the polynucleotide products on the beads within an emulsion. Alternatively, a gene library may be generated followed by next generation sequencing (Steps K, L, K′, and L′).

FIG. 5 is a diagram showing a method of emulsion-based gene synthesis. The illustrated method is amenable to parallel synthesis of many polynucleotides. Steps A-E and associated reagents are consistent with those described in relation to steps A-E of FIG. 4. The diagram shows the orientation of 5′ and 3′ ends during the gene synthesis method. The 5′ and 3′ strand ends are shown in steps G-H, except for strand ‘A’, which is covalently bound to the bead.

FIG. 6 is a diagram showing a method of emulsion-based gene synthesis. The illustrated method is amenable to parallel synthesis of many polynucleotides. As shown in the panel regarding oligo design, the base oligonucleotide ‘A’ template includes, from 3′ to 5′, a T7 RNA polymerase binding site and a variable tag sequence between the identifying sequence and the priming site. Further, the other tile oligonucleotide templates include a variable tag sequence distinct from that of the ‘A’ template between the hybridization sequence and second cleavage site. Steps 1-3 depict on-chip oligonucleotide synthesis and subsequent scission of dsDNA products. In step 4, the products are hybridized to barcoded beads. In step 5, excess reagents are washed away. In step 6, DNA ligase is provided to covalently attach the base ‘A’ oligonucleotide to a corresponding capture oligonucleotide. Step 7 includes contacting the capture complex with one or more of enzymes capable of selectively degrading one strand of one or more tile oligonucleotides. The enzyme may be, for example, T7 exonuclease, UNG, or mcrBC. In step 8, the capture complex is then emulsified with a cleavage reagent capable of cleaving the CS1 cleavage site, liberating the hybridization segments from the identifying sequences of non-base tile oligonucleotides. The emulsion may then be heated and annealed to produce a polynucleotide (steps 9 and 10). Subsequently, as shown in step 11, the emulsion may be broken, the beads washed, and, as shown in step 12, the produced polynucleotides may be treated with ligase. In step 13, the bead-attached polynucleotide may be contacted with T7 RNA polymerase to produce mRNA. The polynucleotides synthesized on the beads may be sequenced by next generation sequencing. From the resultant sequence data, clones with correct sequences may be identified and the variable tag sequences may be read (steps 14 and 15). Further, the produced mRNA may be converted to cDNA and/or used in a PCR reaction with primers complementary to the variable tag associated with the identified correct sequences. The PCR products may be subjected to further Gibson Assembly and error correction.

FIG. 7 is a schematic showing the steps of selectively cloning a nucleic acid molecule having a particular sequence from a pool of nucleic acid molecules having distinct sequences. Step A is the synthesis of nucleic acid molecules, or fragments, that incorporate randomized nucleotides at particular positions at the 5′ and 3′ termini of each molecule, or at the 5′ and 3′ termini of a molecule to be synthesized from the molecules of step A. Step B is the synthesis of a larger nucleic acid molecule from the fragments of step A in an emulsion. Steps A and B may entail, for example, the synthesis of a set of tile oligonucleotides with variable priming sequences incorporated such that emulsion-based synthesis of polynucleotides from the set results in a pool of polynucleotides that includes a plurality of polynucleotides that are identical to or variants of a single sequence of interest and that differ at the variable priming sequence. Step C indicates that the nucleic acid molecules synthesized in the emulsion may be sequenced to determine which variable priming site permutations are associated with a nucleic acid molecule having a desired (“correct”) sequence. As shown in Step D, the correct sequence may then be amplified by PCR using primers specific to the variable priming site permutations flanking that correct sequence. The amplified sequence may then be cloned.

FIG. 8 is a set of four panels (A to D) illustrating steps in the selective amplification of a polynucleotide having a particular sequence from a pool of polynucleotides having distinct sequences. Panel A shows the synthesis of tile oligonucleotides on a chip. Tile oligonucleotides A through H are members of a set of tile oligonucleotides corresponding to a single polynucleotide, with tile oligonucleotides A and H being terminal members of the set. Tile oligonucleotides A and H include 4 randomized nucleotides in a variable priming site, such that the variable priming sites of A and H will flank the sequence of interest in the polynucleotide to be assembled. Panel B shows the assembled polynucleotide in which the variable priming sites flank the sequence of interest and sequencing primer binding sites flank the variable priming sites. Because of the randomized incorporation of base pairs into the variable priming sites, multiple polynucleotides synthesized from the same set of tile oligonucleotides will differ at the variable priming sequences. Panel C shows next generation sequencing of individual strands of the polynucleotides, allowing identification of particular variable priming sequence permutations associated with distinct polynucleotides. In Panel D, primers corresponding to the variable priming sequences of a particular polynucleotide are used to selectively amplify a particular polynucleotide present within the pool. As indicated in Panel D, a polynucleotide having two variable priming sites, each having four randomized base pairs incorporating one of 4 base pairs at each position, provides an arrangement in which 512 distinct primers would be required to prime amplification from all possible combinations of two variable priming sites.

### DETAILED DESCRIPTION

Processes for the production of polynucleotides are of scientific, medical, and commercial value. The present invention is directed to compositions and methods for producing one or more polynucleotides from smaller oligonucleotide segments within an emulsion. In methods of the present invention, a support having one or more capture oligonucleotides is contacted with two or more corresponding tile oligonucleotides. Upon hybridization of the tile oligonucleotides to the capture oligonucleotides, a capture complex is formed. This capture complex is emulsified, optionally with reaction reagents or other additives. The emulsion is then incubated at a temperature regimen sufficient for an adjoining extension reaction to occur, such that a polynucleotide may be formed from the tile oligonucleotides that hybridized to a particular support. A particular advantage of this method is that many different polynucleotides may be produced in parallel with surprising efficiency.

### Polynucleotides

The present invention includes methods for the production of polynucleotides. A polynucleotide may be single-stranded, double stranded, or inclusive of single-stranded and double stranded regions. A polynucleotide of the present invention may be a polynucleotide having a sequence of interest or a variant of a polynucleotide having a sequence of interest. A polynucleotide may further include additional nucleotides located 3′ or 5′ of the sequence of interest on one or both strands. These additional nucleotides may encode, for example, one or more priming sites for amplification of the sequence of interest. The polynucleotide may have DNA nucleobases, RNA nucleobases, modified RNA or DNA nucleobases, synthetic or artificial nucleobases, or a mixture thereof. In particular embodiments, the polynucleotide has only DNA nucleobases. A polynucleotide may be a specific sequence produced, or intended to be produced, in a method of assembling nucleotides. A polynucleotide may include nicks or gaps, provided that the nucleobases of the polynucleotide form a contiguous nucleic acid molecule. A polynucleotide of the present invention may be about 50 bp to 30 kb, or more, in length. For example, a polynucleotide may be about 50 bp, 60 bp, 70 bp, 80 bp, 90 bp, 100 bp, 300 bp, 500 bp, 1 kb, 3 kb, 5 kb, 8 kb, 10 kb, 20 kb, or 30 kb in length.

In some embodiments, the assembled polynucleotide will be a double-stranded DNA molecule. In other embodiments, the assembled polynucleotide will comprise both double-stranded and single-stranded segments of DNA. In particular embodiments, the double-stranded and single-stranded segments of DNA will alternate one or more times along the length of the assembled polynucleotide. The polynucleotide may include nicks. In all cases, a polynucleotide shall be understood to provide the complete sequence of a double-stranded nucleic acid having a sequence consistent with or derived from that of the polynucleotide.

The polynucleotide may optionally include introns, exons, structural sequences, or non-coding regions (e.g., untranslated regions). The polynucleotide may be a gene or gene fragment. It may encode a polypeptide, protein, enzyme, or antibody. A polynucleotide may have a sequence present in the genome of an organism. A polynucleotide may be a variant of a sequence present in the genome of an organism. The organism may be a eukaryote, prokaryote, or archaea. The organism may be a fungus (e.g., a pathogen), bacteria, plant (e.g., a crop plant), or animal. The animal may be a mammal (e.g., a human). A polynucleotide may be an artificial sequence that is not normally present in nature.

### Tile Oligonucleotides

Tile oligonucleotides may be designed to correspond to a polynucleotide of interest or to a variant thereof. A tile oligonucleotide may include a hybridization segment and may further include an identifying sequence, one or more cleavage sites, or additional nucleotides such as a priming sequence, as detailed below. A tile oligonucleotide may additionally include nucleotides that do not serve a function in the present invention or that act as a spacer. The hybridization segment may be a sequence of 10 to 2,000 or more nucleotides. For instance, the hybridization segment may be, e.g., 10, 15, 20, 25, 50, 100, 500, 1,000, 2,000, or more nucleotides. Accordingly, the polynucleotide segment to which the tile oligonucleotide is capable of hybridizing may be a sequence of 10 to 2,000 or more nucleotides. For instance, the segment to which the tile oligonucleotide is capable of hybridizing may be 10, 15, 20, 25, 50, 100, 500, 1,000, 2,000, or more nucleotides. The total length of a tile oligonucleotide may be, for example, 20 to 2,000 or more nucleotides. For instance, the total length of a tile oligonucleotide may be 20, 50, 100, 500, 1,000, 2,000, or more nucleotides. A tile oligonucleotide may be single-stranded or double-stranded. In some instances, the tile oligonucleotide includes both double-stranded and single-stranded segments. A double-stranded tile oligonucleotide may refer to a fully double-stranded oligonucleotide or a double-stranded oligonucleotide with one or two single-stranded overhangs. A set of tile oligonucleotides will include tile oligonucleotides that overlap. The region of overlap between the hybridization segments of any two tile oligonucleotides may be, for example, from 10 base pairs to 2,000 or more base pairs. For example, the region of overlap may be 20, 50, 100, 500, 1,000, 2,000, or more base pairs.

### Identifying Sequences

A tile oligonucleotide of the present invention may include an identifying sequence. An identifying sequence may be present in a single-stranded segment of a tile oligonucleotide. For example, an identifying sequence may be present in a single-stranded overhang of a double-stranded tile oligonucleotide.

The identifying sequence of a tile oligonucleotide may be randomly assigned or assigned from a defined pool of candidate identifying sequences. The identifying sequence of a tile oligonucleotide may be non-randomly assigned. For example, the identifying sequence of a tile oligonucleotide may be capable of hybridizing to a barcode attached to a support (e.g., a bead). Such an identifying sequence can be a complementary barcode (cBarcode), e.g., a sequence complementary to the barcode. In some instances, the identifying sequence will include one or more nucleotides that vary or are randomly selected, while other positions of the identifying sequence do not vary or are non-randomly selected. Typically, one or more tile oligonucleotides will be synthesized such that a particular identifying sequence is associated with one or more particular hybridization segments in a known manner. As a result, in such arrangements, tile oligonucleotides having particular hybridization segments may be isolated or collected by isolating or collecting tile oligonucleotides having a particular identifying sequence.

Identifying sequences may be used to organize tile oligonucleotides into capture complexes. In some instances, all of the tile oligonucleotides of a set corresponding to a particular polynucleotide will share the same identifying sequence. In this way, a single support having capture oligonucleotides of a single corresponding sequence may capture all the tile oligonucleotides of the set. In some instances, two or more distinct sets, each having tile oligonucleotides sharing a single distinct identifying sequence, will be present in a pool of tile oligonucleotides, and two or more supports, each having capture oligonucleotides corresponding to only one set, may be contacted with the pool. In this arrangement, distinct supports may isolate or collect all of the tile oligonucleotides of distinct sets from a single pool. In other arrangements, the pool may include two or more sets having distinct identifying sequences and the pool may be contacted with supports synthesized such that each support corresponds to two or more sets. In such an arrangement a single support will isolate or collect all of the tile oligonucleotides corresponding to two or more polynucleotides. These are merely illustrative examples, as supports corresponding to a single polynucleotide or a plurality of polynucleotides and pools of tile oligonucleotides including one set or a plurality of sets may be combined in any fashion. Supports and polynucleotides may be readily synthesized for any such arrangement according to the methods of the present invention.

Elaborating upon the arrangements described above, it is not required by the present invention that all of the tile oligonucleotides of a set share a common identifying sequence. For instance, each tile oligonucleotide in a set may have a distinct identifying sequence. Alternatively, the number of identifying sequences present in a set of tile oligonucleotides may be more than one but less than the number of tile oligonucleotides in the set. For instances, the total number of distinct identifying sequences present in a set may be 2, 3, 4, 5, 6, 7, 8, 9, 10, or more distinct identifying sequences. In some embodiments, one or more tile oligonucleotides may have two or more identifying sequences. The invention may include any arrangement of the above. Supports with corresponding capture oligonucleotides may be synthesized accordingly for the purposes of the present invention.

The arrangements of the present invention shall not be limited to arrangements involving a small number of polynucleotides, such as 1 to 5 polynucleotides, and a small number of identifying sequences, such as 1 to 5 identifying sequences. The invention shall be understood to also encompass arrangements having many distinct polynucleotides, e.g., thousands, and many identifying sequences, e.g., thousands, as also indicated elsewhere herein. The present invention is well-suited to massively parallel synthesis of polynucleotides. For instance, the pool may include 10², 10³, 10⁴, 10⁵, 10⁶, 10⁷, 10⁸, 10⁵, 10¹⁰, 10¹², 10¹², or more tile oligonucleotides. The tile oligonucleotides may include as many as 10⁵, or more, sets corresponding to distinct polypeptides. For instance, the pool may include 10¹, 10², 10³, 10⁴, 10⁵, or more sets. The sets may correspond to 1 to 100,000 or more distinct polypeptides of interest. For instance, the pool of tile oligonucleotides may include 2, 3, 4, 5, 6, 7, 8, 9, 10, 20, 30, 40, 50, 60, 70, 80, 90, 100, 500, 1,000, 5,000, 10,000, 50,000, 100,000, or more distinct tile oligonucleotides. Contacting the pool with supports corresponding to the sets present in the pool will isolate or collect the tile oligonucleotides required to produce particular polynucleotides of interest.

### Capture Oligonucleotides

A capture oligonucleotide is an oligonucleotide present on a support and capable of hybridizing to at least one identifying sequence of at least one tile oligonucleotide. A capture oligonucleotide can be a barcode, as described herein. The support may be a bead, and the coupling of the capture oligonucleotide to the support may be, e.g., by a biotin-streptavidin interaction. The coupling may be a biochemical or physical interaction. A support may include 1 to 100,000 or more (e.g., 1 to 10,000) individual capture oligonucleotides. For instance, a support may include 1, 2, 5, 10, 50, 100, 500, 1000, 5,000, 10,000, 50,000, 100,000, or more individual capture oligonucleotides. In some examples, a support may have 100,000 to 1,000,000 or more individual capture oligonucleotides, such as 100,000, 200,000, 300,000, 400,000, 500,000, 600,000, 700,000, 800,000, 900,000, 1,000,000 or more individual capture oligonucleotides. In some embodiments, the capture oligonucleotides present on a support may include 2 to 1,000 or more distinct capture oligonucleotides (e.g., 2 to 100). For instance, a support may include 2, 5, 10, 20, 50, 100, 500, 1,000, or more distinct capture oligonucleotides. In particular embodiments, all of the capture oligonucleotides present on a support will be the same.

A support may be synthesized to capture a particular group of tile oligonucleotides. For instance, a support may have capture oligonucleotides corresponding to every member of a set of tile oligonucleotides. In some embodiments, a support may be synthesized to correspond to a single set of tile oligonucleotides. In alternative embodiments, a support may be synthesized to correspond to 2 or more sets of tile oligonucleotides, such as 2 to 30. For instance, a support may include capture oligonucleotides corresponding to 2, 3, 4, 5, 6, 7, 8, 9, 10, 20, or 30 sets of tile oligonucleotides.

The number of sets of tile oligonucleotides a support is designed to isolate is not limiting to the number of capture oligonucleotides that may be present on a support, as a support may comprise numerous capture oligonucleotides of a single sequence. For instance, a support may include a total of 2-100,000 capture oligonucleotides having a particular sequence.

In some embodiments the number of each distinct capture oligonucleotides on a support is the same for each distinct capture oligonucleotide. However, in alternative embodiments, one or more capture oligonucleotides may be present on a support in greater number than one or more other capture oligonucleotides. For example, a support may be synthesized to include a larger number of capture oligonucleotides corresponding to a rare, difficult to capture, or critical tile oligonucleotides. In particular embodiments, a support may have a greater number of a terminal tile oligonucleotide than of other tile oligonucleotides. In these embodiments, the rare, difficult to capture, or critical tile oligonucleotide(s) will include an identifying sequence distinct from at least one other tile oligonucleotide in a set corresponding to the support.

### Barcodes

A barcode of the present invention is a nucleic acid identifier that can be distinguished from other barcodes by its nucleic acid sequence. A barcode can be attached to another molecule, thereby “tagging” the molecule. For example, a barcode can be attached to a support, such as a bead (e.g., a fluorescent bead), or to another nucleic acid. A barcode can further be utilized as a capture oligonucleotide as described herein, for example, to capture a tile oligonucleotide containing an identifying sequence complementary to the barcode. The sequence of a barcode can be randomly assigned or pre-determined (e.g., a sequence complementary to an identifying sequence on a tile oligonucleotide to be captured). A barcode of the invention can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt), or more. In certain embodiments, a barcode can be constructed in combinatorial fashion by combining randomly selected oligonucleotide indexes (e.g., about 1, 2, 3, 4, 5, 6, 7, 8, 9, or 10 indexes).

### Supports

The present invention features supports that can be, for example, attached to one or more capture oligonucleotides (e.g., barcodes) capable of hybridizing to at least a portion of a target nucleic acid (e.g., a tile olignonucleotide). Such supports can thus be used to capture tile oligonucleotides having a particular identifying sequence (e.g., a cBarcode sequence complementary to a barcode attached to the support). Exemplary supports include beads (e.g, Luminex microspheres, magnetic beads), chips, compartments (e.g., tubes, wells, and any other container known in the art). A support can be labeled. Exemplary labels for supports include colored labels (e.g., with a dye) and/or fluorescent labels (e.g., via a fluorescent moiety, such as a fluorescent dye or fluorescent protein as well known in the art). For example, a magnetic bead can be colored or fluorescently labeled. Such labeled beads can be sorted, isolated, or analyzed according to their label. For example, fluorescently labeled beads can be sorted or analyzed using flow cytometry.

A bead attached to one or more barcodes is referred to herein as a “barcoded bead,” and can be used to capture tile oligonucleotides containing an identifying sequence complementary to the barcode. A support can be attached to multiple copies of a particular barcode, or may be attached to a plurality of distinct barcodes. Each barcode on a support can, for example, hybridize to a distinct tile oligonucleotide. In some embodiments, a bead is attached to (i) a barcode capable of capturing a base oligonucleotide, and (ii) a barcode capable of capturing a tile oligonucleotide containing a region overlapping to a portion of the base oligonucleotide. The bead may, in certain embodiments, contain multiple distinct barcodes, each corresponding to a distinct tile oligonucleotide. The bead may contain multiple copies of each of these barcodes. Thus, a bead can, for example, capture the base oligonucleotide and all tile oligonucleotides corresponding to a particular product to be synthesized according to the methods of the invention (e.g., a gene or gene family).

### Synthesis of Tile Oligonucleotides

In some instances, a tile oligonucleotide of the present invention may be synthesized from a single-stranded template oligonucleotide. The template oligonucleotide may include a hybridization segment and a priming site. The template oligonucleotide may further comprise an identifying sequence. In particular instances, the template may additionally comprise one or more cleavage sites.

A tile oligonucleotide may be produced by providing a primer capable of hybridizing to the priming site of a template oligonucleotide and extending the primer, producing a synthesized strand complementary to the template. Various methods of DNA extension, such as PCR and related methods, are known in the art. A plurality of tile oligonucleotides may be synthesized from a single pool including a plurality of templates. The pool may include or be synthesized to include similar or identical amounts of each template. Alternatively, the pool may include or be synthesized to include more of one or more templates than other templates. The pool may be contacted with primers provided or mixed to be provided in similar or identical amounts. Alternatively, the pool may be contacted with a plurality of primers provided or mixed to be provided such that one or more primers is provided in excess of one or more other primers.

Following extension, the newly synthesized strand may be disassociated from the template oligonucleotide and may be used as a single-stranded tile oligonucleotide. In alternative embodiments, the template oligonucleotide and synthesized strand together are a double-stranded tile oligonucleotide. In some instances, the template strand is longer than the newly synthesized strand, such that the double-stranded tile oligonucleotide includes one or two single-stranded overhangs. For instance, if the priming site is 5′ of the 3′ terminus of the template oligonucleotide, extension from the priming site will produce a double-stranded oligonucleotide having a 3′ overhang, the overhang being made up of template strand nucleotides that extend beyond the 5′ end of the newly synthesized strand. In particular examples, an identifying sequence is present in the 3′ overhang. In alternative examples, the primer capable of hybridizing to the template oligonucleotide includes a 3′ segment capable of hybridizing to the priming site of the template oligonucleotide and an identifying sequence 5′ of that segment, such that the identifying sequence does not hybridize to the template oligonucleotide.

Synthesis of a tile oligonucleotide from a template oligonucleotide may take place by solid state synthesis. In solid state synthesis methods, the template oligonucleotide is affixed to a solid state synthesis structure. In some embodiments the template oligonucleotide is affixed to the solid state synthesis structure by its 5′ terminus. In particular embodiments, an identifying sequence will be 3′ of the hybridization sequence of the template oligonucleotide. The template oligonucleotide may be contacted with a primer and extension may occur from the template oligonucleotide while the template oligonucleotide is affixed to the synthesis structure. In these particular methods, the template oligonucleotide will encode a cleavage site positioned on the template such that cleavage at the cleavage site subsequent to extension of the primer will separate the template and the newly synthesized strand from the synthesis structure. In such embodiments, the cleavage site is positioned such that the hybridization segment encoded by the template is released from the synthesis structure upon cleavage. For instance, if the 5′ terminus of the template is affixed to the synthesis structure, the cleavage site must be 3′ of the hybridization segment. Following extension of the primer, a cleavage agent capable of cleaving the cleavage site may be contacted with the solid state synthesis structure, thereby liberating the tile oligonucleotide from the structure.

In alternative solid state synthesis arrangements, the template oligonucleotide may be affixed to the synthesis structure at its 3′ terminus. Extension occurs from the template oligonucleotide while the template oligonucleotide is affixed to the synthesis structure. In these particular methods, as above, the template encodes a cleavage site positioned on the template such that cleavage at the cleavage site subsequent to extension of the primer will separate the template and the newly synthesized strand from the synthesis structure. Here, the cleavage site may be 3′ of the identifying sequence and hybridization segment of the template oligonucleotide. Following extension of the primer, a cleavage agent capable of cleaving the cleavage site may be contacted with the solid state synthesis structure, thereby liberating the tile oligonucleotide from the structure.

In alternative embodiments, a template oligonucleotide priming sequence and identifying sequence may be positioned on opposite sides of the hybridization segment. For instance, the identifying sequence may be 5′ of the hybridization sequence and the priming site may be 3′ of the hybridization sequence on the template oligonucleotide. In these embodiments, a mechanism of truncating extension may be present in the template oligonucleotide between the hybridization sequence and the identifying sequence such that the identifying sequence will be single-stranded in the resulting double-stranded tile oligonucleotide. The mechanism of truncating extension may be a non-native base, such as a base other than an A, C, G, or T. The non-native base may be positioned immediately adjacent to the most 3′ nucleotide of the identifying sequence or elsewhere between the hybridization sequence and the identifying sequence. In these particular methods of solid phase synthesis, the template oligonucleotide may be affixed to the synthesis structure by either the 5′ or 3′ terminus. The template may further include a cleavage site for liberation of the tile oligonucleotide from the synthesis structure.

Upon cleavage of a template oligonucleotide and an associated newly synthesized strand from a synthesis structure, the liberated segment of the template strand and newly synthesized strand are a double-stranded tile oligonucleotide. In some instances, cleavage will sever a segment capable of hybridizing to a polynucleotide. In these instances, the liberated portion of the segment capable of hybridizing to a polynucleotide is the hybridization segment.

Alternatively, a newly synthesized strand may be decoupled from the template strand, for instance by incubation at a denaturing temperature. The newly synthesized strand may be subsequently converted to a double-stranded tile oligonucleotide by a strand-displacing polymerase. In such embodiments, a displacement priming site present on the newly synthesized strands is contacted with a complementary primer. A nick may be enzymatically introduced into the primer by a cleavage reagent. The primer may then be extended by a strand-displacing polymerase in the presence of appropriate reagents and under a temperature regimen conducive to extension of the primer by the strand-displacing polymerase. In certain embodiments, the extension is limited to a single round of extension, such that the primer is extended but subsequent amplicons are not generated. In these embodiments, the strand produced by the strand-displacing polymerase, the displacement strand, will remain hybridized to the newly synthesized strand that served as the template in this reaction, forming a double-stranded tile oligonucleotide. Methods of extension using a strand-displacing polymerase are known in the art.

Production of a tile oligonucleotide including a newly synthesized strand and a displacement strand may be synthesized from a template oligonucleotide affixed to a synthesis structure. In some embodiments, the template oligonucleotide will be affixed to the synthesis structure at its 3′ terminus. In alternative embodiments, the template oligonucleotide will be affixed to the synthesis structure at its 5′ terminus. In certain embodiments, the template oligonucleotide will include a priming sequence 3′ of the hybridization sequence, a displacement priming sequence 5′ of the hybridization sequence, and an identifying sequence 5′ of the displacement priming sequence. In these embodiments, extension from the priming sequence will produce a newly synthesized strand and that strand will be released from the template oligonucleotide. For instance, the newly synthesized strand may be released by incubation of the synthesis structure at a denaturing temperature, after which the released newly synthesized strands may be optionally washed, isolated, or purified. The newly synthesized strands may then be contacted with one or more primers capable of hybridizing to the displacement priming sites and incubated with reaction reagents including a strand-displacing polymerase at a temperature conducive to extension by the strand-displacing polymerase. This will produce a double-stranded tile oligonucleotide. In particular embodiments, extension by the strand-displacing polymerase will produce a double-stranded tile oligonucleotide in which the 3′ end of the newly synthesized strand includes a single-stranded 3′ overhang that extends beyond the 5′ end of said displacement strand and includes an identifying sequence.

In some embodiments, the solid state synthesis structure includes 1 to 10⁵ or more template oligonucleotides. For instance, the solid state synthesis structure may include 1, 10², 10³, 10⁴, 10⁵, 10⁶, 10⁷, 10⁸, 10⁵, or more template oligonucleotides. In some embodiments, each template has the same cleavage site. In alternative embodiments, the templates include 2 or more distinct cleavage sites, such as 2-20 distinct cleavage sites. In some embodiments, the cleavage sites are enzymatic cleavage sites and the cleavage agent is an enzyme. Numerous cleavage sites and corresponding cleavage enzymes are known in the art.

Double-stranded tile oligonucleotides may also be generated by the fusion of two separately synthesized strands. For instance, two template oligonucleotides may be used to synthesize complementary or partially complementary strands, with subsequent liberation of both newly synthesized strands from their respective templates followed by hybridization of the two newly synthesized strands to each other, forming a tile oligonucleotide.

A significant advantage of double-stranded tile oligonucleotides is the reduction in the potential for cross-hybridization. However, alternative or additional methods of minimizing cross-hybridization are available. For instance, a double-stranded tile oligonucleotide may be contacted with a strand displacing polymerase and a short single-stranded nucleic acid to block cross-hybridization to other tile oligonucleotides or mismatched sequences. In some methods, a strand of a tile oligonucleotide may include ‘extra’ non-terminal nucleotides such that the strand must form a hairpin in order for the non-hairpin nucleotides to fully hybridize to an otherwise complementary sequence. In particular examples, the hairpin-containing strand and a complementary strand are synthesized from separate template oligonucleotides, released from the template oligonucleotides, and subsequently contacted with each other for hybridization. In some instances, the identifying sequence of a tile oligonucleotide strand may be of greater length or annealing strength than the hybridization sequence or of the remainder of the tile oligonucleotide strand. Tile oligonucleotides within a pool of tile oligonucleotides may be synthesized using a strategy of codon or nucleotide selection that reduces or minimizes the likelihood of two mismatched tile oligonucleotide strands within a pool cross-hybridizing. In this regard, wobble bases may be incorporated into the design of the polynucleotide to inhibit cross-hybridization. The above methods may be combined in all possible combinations.

In still other mechanisms of inhibiting cross-hybridization of mismatched tile oligonucleotide strands, a tile oligonucleotide may include a 5′ methylC adjacent to the identifying sequence such that contacting the tile oligonucleotide to DpnI will nick the DNA.

Any of the above methods of synthesis may be optionally improved by controlling depurination. Controlling depurination, and particularly reducing depurination, may improve the fidelity and efficacy of tile oligonucleotide synthesis. Methods of synthesis may be further improved by matching the melting temperatures of priming sequences or template tile oligonucleotides synthesized in parallel, such as tile oligonucleotides synthesized from a single synthesis structure.

### Capture Complex

A capture complex is formed when one or more capture oligonucleotides present on a support (e.g., a barcode attached to a bead) hybridize to one or more tile oligonucleotides, or a portion thereof (e.g., an identifying sequence complementary to the barcode). In some embodiments, a support may be contacted with a pool containing tile oligonucleotides of a single set. In alternative embodiments, a support may be contacted with a pool containing tile oligonucleotides of 2 to 10⁵, or more, sets corresponding to distinct polypeptides. For instance, the pool may include 10¹, 10², 10³, 10⁴, 10⁵, or more sets corresponding to distinct polypeptides that may be produced by a method of the present invention in a single emulsion. In some instances, a pool of tile oligonucleotides will be contacted with one or more supports corresponding only to a single set. In other instances, a pool of tile oligonucleotides may be contacted with 2 to 10,000 or more distinct supports corresponding to a plurality of sets. In these embodiments, the number of distinct supports may be 2, 5, 10, 50, 100, 500, 1,000, 5,000, 10,000, or more. Thus, in the methods of the present invention, a plurality of supports may be contacted with a pool containing a plurality of tile oligonucleotides, whereby each support may capture tile oligonucleotides to which it corresponds. In doing so, capture complexes are formed that collect or isolate particular sets of tile oligonucleotides.

Conditions may be optimized for efficient hybridization. For example, recA protein or peptide may be present, or a crowding agent such as polyethylene glycol, fusaric acid, or similar crowding agents may be present. Incubation temperatures or temperature regimens may also be optimized with respect to the selected method and the tile oligonucleotides and capture oligonucleotides at hand. Methods of optimizing hybridization efficiency are known in the art.

### Emulsion and Tile Oligonucleotide Liberation

An emulsion may be a means of compartmentalizing a set of reagents or a reaction involving a set of reagents. The present invention may include emulsification of one or more capture complexes. In order for the hybridization segments associated with a capture complex to form a polynucleotide, one, two or more, all but one, or all of the distinct hybridization segments in a set must be liberated from the support. Emulsification allows hybridization segments associated with a capture complex to remain isolated and co-localized when released from the capture complex for the purpose of forming a polynucleotide. Emulsification can be used to produce microreactors (e.g., in emulsion droplets 100 μm in diameter or smaller) useful for, e.g., amplification of nucleic acid species (e.g., by emulsion PCR). Such amplification reactions can be performed, for example, with at least 10⁸ microreactors per microliter of reaction mixture.

Emulsion may be achieved by a variety of methods known in the art. In some embodiments, the emulsion is an emulsion that is stable to a denaturing temperature, e.g., to 95° C. or higher. An exemplary emulsion may be an oil and water emulsion. In some embodiments, the emulsion may be a perfluorcarbon oil emulsion (e.g., a water-in-perfluorocarbon oil emulsion). A water-in-perfluorocarbon oil emulsion may be highly stable, such that the emulsion microcapsules can be stored for years with little, if any, exchange of gene products between microcapsules. Synthesis of an emulsion generally requires the application of energy (e.g., mechanical energy) to force the phases together. Methods for generating emulsions may include use of mechanical devices (e.g., stirrers, homogenizers, colloid mills, ultrasound, and membrane emulsification devices). For example, mechanical agitation can be performed using a vortex Genie. A single constituent, such as a bead (e.g., a barcoded bead), can be encapsulated within an emulsion microdroplet, for example, by statistical loading, which generally involves producing an excess of emulsion microdroplets compared to the number of constituents (e.g., 10 times more microdroplets than beads). Alternatively, encapsulating single constituents (e.g., barcoded beads) within emulsion microdroplets can be achieved by making microdroplets small enough that only a single constituent can fit within each microdroplet.

An emulsion may be a well or a plurality of wells in which one or more capture complexes are compartmentalized. Compartmentalization of capture complexes into wells may be achieved, in some embodiments, due to physical limitations relating to the mass or dimensions of the capture complexes, the dimensions of the well, or a combination thereof. A well may be a fiber-optic faceplate where the central core is etched with an acid, such as an acid to which the core-cladding is resistant. A well may be a molded well. The wells may be covered to prevent communication between the wells, such that the beads present in a particular well remain within the well or are inhibited from moving into a different well. The cover may be a solid sheet or physical barrier, such as a neoprene gasket, or a liquid barrier, such as perfluorocarbon oil.

An emulsion of the present invention may be a monodisperse emulsion or heterodisperse emulsion. Each droplet in the emulsion may contain, or contain on average, 0-10 supports. For instances, a given droplet may contain 0, 1, 2, 3, 4, 5, 6, 7, 8, 9, or 10 supports. In particular embodiments, a given droplet may contain 0, 1, 2, or 3 supports. On average, the droplets of an emulsion of the present in invention may contain 0-3 supports, such as 0, 1, 2, or 3 supports, as rounded to the nearest whole number. In some embodiments, the number of supports in each emulsion droplet, on average, will be 1, or between 0 and 1, or between 1 and 2.

The emulsion of the present invention may include various compounds, enzymes, or reagents in addition to the capture complex and emulsion media of the present invention. These additives may be included in the emulsion solution prior to emulsification. Alternatively, the additives may be added to individual droplets after emulsification. Exemplary additives include cleavage enzymes, SO-PCR reagents, emulsion PCR reagents, and reagents for Gibson Assembly.

Within the emulsion droplet, one or more of the captured tile oligonucleotides must be liberated from the capture complex in order for formation of the polynucleotide to occur. In some embodiments, one or more tile oligonucleotides are liberated from the capture complex by incubation at a denaturing temperature. In other embodiments, the mechanism of liberation involves cleavage of the tile oligonucleotide. In these embodiments, one or more of the tile oligonucleotide present in the capture complex must comprise a cleavage site. This cleavage site must be positioned between the support and a hybridization segment of the tile oligonucleotide, in particular separating the identifying sequence from the hybridization sequence. In these embodiments, the emulsion will further include one or more cleavage agents capable of cleaving one or more cleavage sites present on one or more captured tile oligonucleotides. In some exemplary embodiments, the cleavage site may be an enzymatic cleavage site and the cleavage agent may be an enzyme. In particular embodiments, the cleavage site may be a single stranded region that is cleaved by a nicking enzyme. Once liberated, one or more liberated hybridization segments may hybridize to one or more other hybridization segments that are either similarly liberated or that remain on the support.

Within the emulsion droplet, in certain embodiments, the identifying sequence of the tile oligonucleotide is separated from the hybridization segment. As described above, cleavage of the identifying sequence may be employed as a mechanism of liberating the hybridization segment from the capture complex. In various embodiments, however, the tile oligonucleotide may be liberated from the capture support by incubation at a denaturing temperature. In such embodiments, the identifying sequence of the tile oligonucleotide may yet incorporate a cleavage site positioned between the hybridization segment and the identifying sequence. In these embodiments, the emulsion will include a capture reagent capable of cleaving this cleavage site and the emulsion will be incubated at a temperature sufficient to promote cleavage. In alternative embodiments, the identifying sequence is part of the hybridization segment.

### Methods of Adjoining Extension

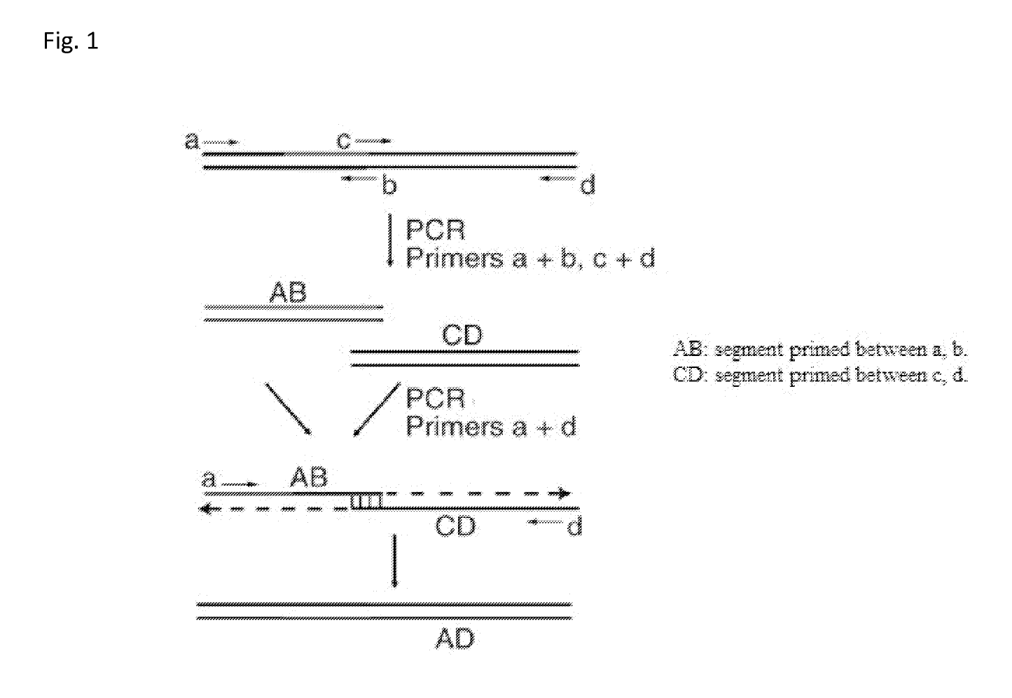

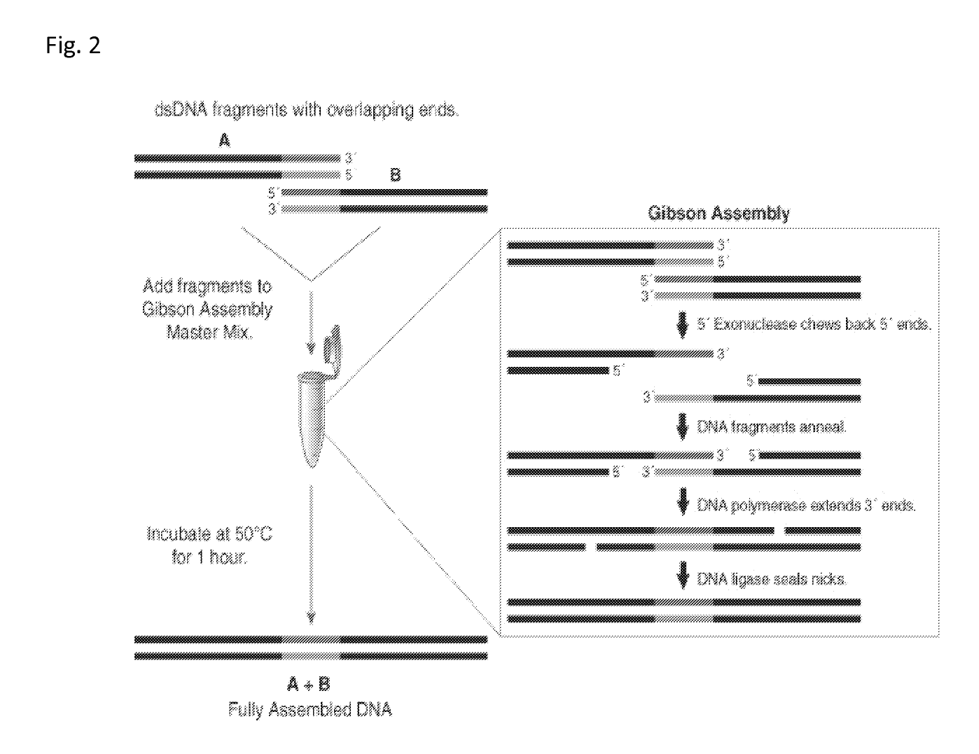

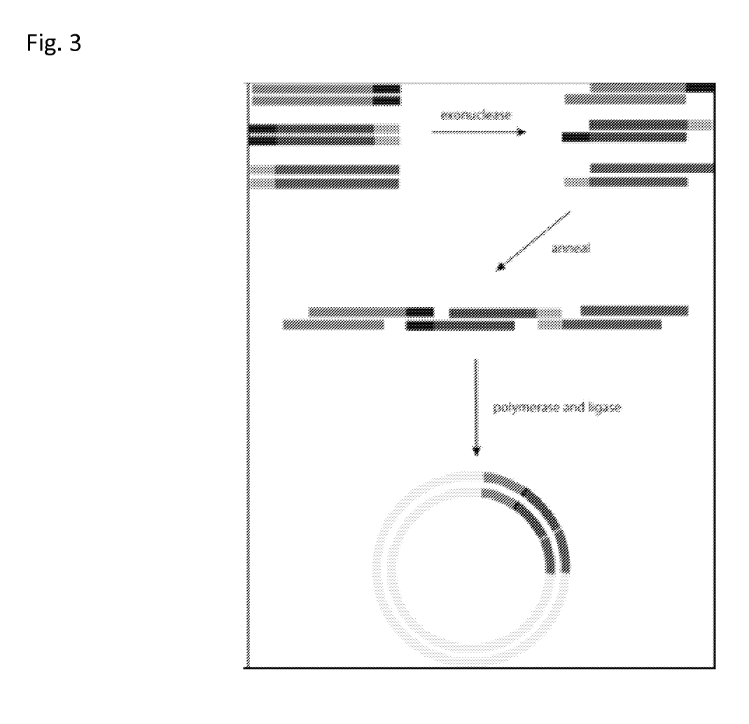

Adjoining extension reactions are known in the art. Exemplary adjoining extension reactions include splice-overlap PCR (SO-PCR; FIG. 1) and Gibson Assembly (FIGS. 2, 3). SO-PCR is a versatile technique for site-directed mutagenesis and gene splicing. Overlapping gene segments are used as template DNA for PCR to create a full-length product. Internal primers generate overlapping, complementary 3′ ends on the intermediate segments and may be used to introduce nucleotide substitutions, insertions or deletions for site-directed mutagenesis, or for gene splicing. Segments encode the nucleotides found at the junction of overlapping, complementary gene segments. Overlapping strands of these intermediate products hybridize at this 3′ region in a subsequent PCR and are extended to generate the full-length product amplified by flanking primers that can include restriction enzyme sites for inserting the product into an expression vector for cloning purposes. The highly efficient generation of mutant or chimeric genes by this method may be accomplished with standard laboratory reagents.

Gibson Assembly is a commonly used in vitro assembly method, as it is facile, flexible and may be readily optimized. Gibson Assembly requires overlapping, complementary oligonucleotides and three enzymatic reaction reagents, cumulatively present in the Gibson Assembly Master Mix. DNA fragments may be contacted with the master mix and incubated at a temperature regimen sufficient to carry out Gibson Assembly, e.g., 50° C. for 1 hour. The resulting product is a double-stranded polynucleotide suitable for a range of downstream applications.

Other methods of adjoining extension may also be used.

A method of adjoining extension may require 0, 1, 2, 3, 4, 5, 10, 15, or 20 reagents. Similarly, a temperature regimen may be selected that is sufficient for practicing a selected method of adjoining extension. Temperature regimens appropriate to numerous adjoining extension reactions are known in the art.

The various steps of the present invention may be used together in any order capable of producing a polynucleotide of the present invention. Conditions may be optimized for efficient hybridization. For example, recA protein or peptide may be present, or a crowding agent such as polyethylene glycol, fusaric acid, or similar crowding agents may be present.

### Base Oligonucleotides

In particular embodiments, it is desirable that a polynucleotide produced within an emulsion droplet is attached to a support. For instance, if the polynucleotide is attached to the support, the polynucleotide may be isolated by a method of isolating the support to which it is attached. This may be achieved if, throughout adjoining extension, a terminal hybridization segment of a tile oligonucleotide corresponding to the polynucleotide remains attached to the support. In certain embodiments, the tile oligonucleotide that remains attached to the support is a base oligonucleotide.

In particular methods, a base oligonucleotide may be synthesized by any template-based synthesis method discussed herein, with the addition that the primer that hybridizes to the template includes a 5′ phosphate. Like other tile oligonucleotides, a base oligonucleotide may hybridize to a corresponding capture oligonucleotide. The base oligonucleotide and capture oligonucleotide will be synthesized such that interaction of the capture oligonucleotide and the identifying sequence of the base oligonucleotide results in abutment of the 5′ phosphate with the free 3′ terminus of the capture oligonucleotide, resulting in a nick structure. A capture complex having hybridized a base oligonucleotide in this manner may then be treated with ligase, covalently joining the 5′ end of the newly synthesized strand of the base oligonucleotide to the 3′ end of the capture oligonucleotide with which it corresponds. The capture complex contacted with ligase may be incubated with any additional reagents and at a temperature regimen sufficient to promote ligation. Ligation may occur prior to emulsion or within an emulsion. In emulsion, the base oligonucleotide will be incorporated into a corresponding polynucleotide. As a result, the polynucleotide will be attached to the support.

In alternative methods, the base oligonucleotide may be a tile oligonucleotide that lacks a cleavage site that is present on one or more other oligonucleotides corresponding to the same polynucleotide. For instance, in such a method, the uncleaved base oligonucleotide remains attached to the support while other, cleaved tile oligonucleotides are liberated. The corresponding polynucleotide is therefore produced such that it is attached to the bead.

In some embodiments, a set of tile oligonucleotides has one base oligonucleotide. In other embodiments, a set of tile oligonucleotides has two base oligonucleotides. In still others, a set of tile oligonucleotides has no base oligonucleotide.

### Degrading a Strand of the Tile Oligonucleotide

In some methods of the present invention, it is desirable to reduce the complexity of the nucleic acids present within emulsion droplets. For instance, in particular methods it may be desired to selectively degrade one strand of one or more tile oligonucleotides. Typically, DNA hydrolysis is not strand-specific. A variety of methods may be used to achieve strand-specific degradation. In the present invention, the selective degradation of a single strand of a tile oligonucleotide is possible, for example, if one strand of the tile oligonucleotide is a protected strand while the second strand is a non-protected strand. The present invention may provide an enzyme, compound, or chemical that selectively degrades a non-protected strand over a protected strand. A tile oligonucleotide having a protected strand and a non-protected strand may be denatured and treated with such an enzyme, compound, or chemical.

A strand may include modified nucleobases or nucleobases other than adenine, guanine, cytosine, and thymine, such that particular enzymes may selectively degrade a strand with or a strand without a particular modification or nucleobase. A modification or atypical base present in one strand may be fully or only partially absent from the other. For example, three or more 5′ phosphorothioates may protect a strand from degradation. In contrast, a strand with zero, one, or two phosphorothioates will not be protected. A tile oligonucleotide in which the protected strand includes three or more 5′ phosphorothioates and the non-protected strand includes less than three 5′ phosphorothioates may be treated with an enzyme that selectively degrades strands with less than three 5′ phosphorothioates. Examples of exonuclease enzymes capable of selectively degrading strands with less than three 5′ phosphorothioates include T7 exonuclease and lambda exonuclease.

In another example, the protected strand is a nucleotide that includes a 5′ phosphate, whereas the non-protected strand does not include a 5′ phosphate. Lambda exonuclease is an example of an enzyme that may selectively degrade a strand lacking a 5′ phosphate over a stand with a 5′ phosphate.

In one example, an unprotected tile oligonucleotide strand includes methylated adenine nucleobases and the denatured tile oligonucleotide is treated with the enzyme DpnI, which selectively degrades methylated adenine nucleobases. In another example, the protected tile oligonucleotide strand, but not the unprotected tile oligonucleotide strand, includes methylated nucleobases and the denatured tile oligonucleotide is treated with the enzyme Sau3AI, which selectively degrades non-methylated DNA. In still another example, the protected tile oligonucleotide strand, but not the unprotected tile oligonucleotide strand, includes one or more methylated cytosine nucleobases and the denatured tile oligonucleotide is treated with the heterodimeric enzyme mcrBC, which selectively degrades strands with methylated cytosine nucleobases (e.g., (G/A)^{m}C sites on DNA). A further example may include the incorporation of uracil into a non-protected strand, but not a protected strand, with subsequent treatment with an enzyme capable of selectively degrading a uracilated strand over a non-uracilated strand. For example, uracil-DNA glycosylase enzymes degrade uracilated strands of nucleic acids. Other methods of selectively modifying a strand to enable selective degradation are known in the art.

In any of the above examples, the modification or atypical base present in one strand may be fully or only partially absent from the other. In some embodiments the protected strand is the newly synthesized strand of a tile oligonucleotide synthesized from a template and including the template. The non-protected strand may be the template strand. In some embodiments the protected strand is the template strand of a tile oligonucleotide synthesized from a template and including the template. The non-protected strand may be the newly synthesized strand. Selective degradation of the non-protected strand may occur prior to emulsion. Alternatively, selective degradation of the non-protected strand may occur within an emulsion.

Additional Functional Sequences that May be Incorporated into a Polynucleotide

A polynucleotide may include nucleotides 3′ or 5′ of the sequence of interest on one or both strands. A polynucleotide may include, for example, an amplification priming sequence, variable priming sequence, or RNA polymerase binding site 5′ or 3′ of the sequence of interest on one or both strands of the polynucleotide. When present, these additional sequences will be incorporated by inclusion in a tile oligonucleotide of the set of tile oligonucleotides from which the polynucleotide is assembled. In some embodiments, the additional sequence will be incorporated from a terminal tile oligonucleotide, though an additional sequence may also or alternatively be present in a non-terminal tile oligonucleotide.

When present, a single amplification priming sequence may be shared by all of the polynucleotides present in an emulsion or by a subset of polynucleotides present in an emulsion, such as all the polynucleotides present in an emulsion that are synthesized to encode a particular sequence of interest. An amplification priming site may be, for example, 6 to 50 nucleotides (e.g., 15-25 nucleotides) or more, such as 6, 8, 10, 15, 20, 25, 30, 35, 40, 45, 50, or more nucleotides. An amplification priming site will enable amplification of all or a selected subset of polynucleotides present in an emulsion.

In some instances, the amplification priming sequence will be a variable priming sequence. A variable priming sequence means a priming sequence that includes one or more positions that will be synthesized such that two polynucleotides synthesized to have otherwise identical sequences will differ at these variable positions. A variable priming sequence may include only variable nucleotide positions or may include a combination of variable and constant nucleotide positions. For instance, a variable priming sequence may include 1-50 variable nucleotide positions, such as 1, 2, 3, 4, 5, 10, 15, 20, 25, 30, 35, 40, 45, or 50 variable nucleotide positions. In any of these embodiments, the remaining positions in the variable priming sequence will be constant. A variable priming sequence may also be longer than 50 nucleotides. Insofar as the number of nucleotide positions included in the priming sequence is not strictly limited, any given variable priming sequence may include from 0.1% to 100% variable nucleotide positions, such as 0.1%, 1%, 2%, 3%, 4%, 5%, 10%, 15%, 20%, 25%, 30%, 35%, 40%, 45%, 50%, 60%, 70%, 80%, 90%, or 100% variable nucleotide positions. In any arrangement having both constant and variable nucleotide positions, the constant or variable positions may be respectively contiguous or non-contiguous. If non-contiguous, the constant or variable positions, respectively, may be regularly or irregularly dispersed throughout the variable priming sequence.

The variable priming sequences may be synthesized such that a given variable position may be filled by one of any two, three, four, or five of adenine, guanine, cytosine, thymine, and uracil. In other embodiments, the variable positions may also or alternatively be filled by one or more artificial, synthesized, modified or unnatural nucleotides, or any nucleotide other than unmodified adenine, guanine, cytosine, thymine, or uracil. In some instances, an artificial nucleotide will pair with an unmodified, natural nucleotide. In others, however, an artificial nucleotide will selectively pair with another artificial nucleobases to form an artificial base pair. An exemplary artificial base pair may be a 3-fluorobenzene self-pair, a dSICS and dMMO2 pair, a d5SICS and dMMO2 pair, or a d5SICS and dNaM pair. The variable priming sequences may be synthesized to include any combination of adenine, guanine, cytosine, thymine, uracil, and alternative or synthetic nucleobases. The identity of the nucleotide options selected to fill one variable position of a variable priming sequence will not bear upon the nucleotide options that may be selected to fill any other variable position within the same variable priming sequence or otherwise. Any subset of available natural, unnatural, modified, synthetic or artificial nucleotides may be provided independently to fill any particular position.

The nucleotides selected to fill a particular variable position may be provided in equal molar proportions. Alternatively, one or more of the selected nucleotides may be provided in excess to one or more other selected nucleotides. In some embodiments, a variable priming sequence will be synthesized to incorporate a nucleotide from the available or selected nucleotide possibilities in an essentially randomized manner. In others, incorporation of one or more of the selected nucleotides will be favored over the incorporation of one or more other selected nucleotides.

Upon synthesis of a nucleic acid molecule having a variable priming sequence, such as a tile oligonucleotide, the sequence of the variable priming sequence will become determined. Insofar as related variable priming sequences will vary at the variable positions, any particular determined arrangement of nucleotides for a variable priming sequence will be referred to as a permutation. The number of distinct permutations will be a function of the number of variable positions and the number of possible nucleotides.

In some embodiments, the polynucleotide is synthesized to include an RNA polymerase biding site or a fragment thereof. For instance, a polynucleotide may include a T7 RNA polymerase binding site or fragment thereof. In some embodiments, a polynucleotide will comprise one or more variable priming sites and one or more additional amplification priming sites that are not variable priming sites. In certain embodiments, a polynucleotide will comprise one or more variable priming sites and one or more additional RNA polymerase sites. Other combinations are also contemplated.

### Post-Emulsion Procedures

Upon completion of the adjoining extension reaction, the emulsion may be broken. The broken emulsion includes the supports and any polynucleotides produced in the emulsion. In some instances, one or more polynucleotides are attached to a support. Alternatively, a polynucleotide may be free of a support. In particular embodiments, a polynucleotide may be attached to a support but include a cleavage sequence such that the polynucleotide may be subsequently liberated from the support.

In some embodiments, the polynucleotide will include nicks. In these embodiments, the polynucleotide may be further incubated with ligase to covalently seal the nicks. The polynucleotide contacted with ligase may be incubated with any additional reagents and at a temperature regimen sufficient to promote ligation.

In embodiments in which a polynucleotide remains attached to a support after the emulsion is broken, the polynucleotide may be isolated or collected via a detectable label present on the support. For instance, the support my include a dye label, fluorescent label, radio label, electrical conductance signal, fluorescence polarization signal, oligonucleotide label, or mass spectrometric label, or be of a particular size or shape. Examples of detectable labels further include Luminex or GnuBio labels in which ratios of squalene-type dyes or other dyes provide differentiating properties. Many detectable labels are known in the art, including many which may be present on a solid support, such as a bead (e.g., a Luminex bead). Beads useful in the methods of the invention may include differentially-dyed beads (e.g., Luminex beads) that can be analyzed by flow cytometry. Such beads can further be attached to oligonucleotide barcodes to produce barcoded beads, such as those utilized in the methods described herein. Supports may be sorted by a technique appropriate to the label or labels with which the supports are associated. Methods of sorting may include fluorescence-activated cell sorting (FACS), size separation, magnetic separation, charge separation, affinity purification, or other means known in the art. Supports isolated or collected following the breaking of the emulsion may be washed or deposited into individual wells of a microtiter plate. Supports may be deposited to individual wells of a microtiter plate, or each well may include a plurality of supports.

Notably, the polynucleotides generated by the methods of the invention can, themselves, be used in a further emulsion-based synthesis reaction. Here, one or more polynucleotides generated in a first round of synthesis may be used as tile oligonucleotides in a second round of synthesis. Polynucleotides for use in the second round of synthesis may be isolated or collected using capture supports, as described above. Polynucleotides for use in the second round of synthesis may be separated into sets by sorting the first round supports as described above (e.g., by fluorescent sorting of beads). By these approaches, polynucleotides of virtually any length may be generated.

In embodiments in which the polynucleotide is attached to the support after the breaking of the emulsion, the base oligonucleotide may further include a (uncleaved) cleavage site, such as a cleavage site corresponding to a cleavage reagent to which the base oligonucleotide has not been exposed. The base oligonucleotide may further be contacted with this cleavage reagent to separate the polynucleotide from the support.

In some embodiments, one or more polynucleotides will include one or more RNA polymerase sites, amplification sequences, or variable amplification sequences. When present, an RNA polymerase site may be used to produce RNA amplicons from one or both strands of a polynucleotide. An RNA polymerase may be provided that is capable recognizing the RNA polymerase site. For instance, if the RNA polymerase site is a T7 RNA polymerase site, T7 RNA polymerase will transcribe RNA strands from the polynucleotide in the presence of additional amplification reagents and a temperature regimen conductive to this transcriptional amplification. Many copies of a polynucleotide may be produced by the RNA polymerase. Incorporating an RNA polymerase site into one or more polynucleotides and subsequently providing a corresponding RNA polymerase with amplification reagents and a temperature regimen conducive to transcription can effectively produce numerous RNA amplicons of a plurality of polynucleotides.

In some embodiments, one or more polynucleotides will include a non-variable amplification sequence. These polynucleotides may be amplified by providing a corresponding amplification primer, a DNA polymerase, and additional amplification reaction reagents at a temperature regimen conducive to amplification. Methods of amplification are well known in the art. In some embodiments of the present invention, amplification provides a method of producing amplicons of polynucleotides generated within an emulsion.

In some embodiments, one or more polynucleotides will include one or more variable amplification sequences. Polynucleotides present within a pool of polynucleotides may include two or more distinct permutations of one or more variable amplification sequences. Variation at variable positions present in one or more polynucleotides will permit selective amplification of particular polynucleotides over others. In particular, variation at variable positions present in polynucleotides that are otherwise identical will enable selective amplification of particular polynucleotides present within a pool of polynucleotides, including those that may have been synthesized from the same set of tile oligonucleotides. For example, not all polynucleotides synthesized to have a particular sequence will have exactly that sequence. Some will be intentional or unintentional variants of the sequence encoded by the corresponding set of tile oligonucleotides. In particular methods of the present invention, polynucleotides determined to have a particular sequence may be amplified over others that do not by amplification from the variable priming sequence. The invention therefore provides a method of selectively amplifying one or more polynucleotides present in a pool of polynucleotides. One or more permutation-specific primers and reagents for amplification may be used to prepare an amplification reaction that, upon incubation at a temperature regimen conducive to amplification, will selectively amplify only polynucleotides having the variable priming sequence permutation that corresponds to the permutation-specific primers.

In further embodiments, selective amplification of polynucleotides having a particular variable priming sequence permutation will follow a less specific amplification. The less specific amplification may be an RNA polymerase amplification or DNA polymerase amplification from one or more non-variable amplification priming sites. In these embodiments, one or more polynucleotides present in a pool of polynucleotides will have one or more variable amplification priming sequences in addition to one or more RNA polymerase sites or non-variable amplification priming sequences. In these embodiments, at least one of the RNA polymerase or non-variable amplification sites may be 3′ of at least one variable priming sequence such that amplicons produced by amplification from the RNA polymerase or non-variable amplification site will include the variable priming sequences.

Following the first amplification, the pool will include amplicons of a plurality of polynucleotides present within the pool. A subset of these amplicons may be removed from the pool for sequencing. Alternatively, the polynucleotides may be removed from the pool for sequencing. In still other embodiments, a combination of polynucleotides and amplicons may be removed from the pool for sequencing. In particular embodiments, the polynucleotide will remain attached to the bead and may be isolated from the amplicons. In these embodiments the bead-attached polynucleotides, free amplicons, or a combination thereof may be used for sequencing. Sequencing may be accomplished by any method known in the art and may be a method of deep sequencing. Sequencing may reveal the sequence of particular polynucleotides, including the sequence of one or more variable amplification sites associated with the sequence of interest. These polynucleotides may be selectively amplified from the remaining pool of amplicons, polynucleotides, or amplicons and polynucleotides by targeting the identified permutation of the variable amplification site. In these embodiments, a permutation-specific primer will be contacted with the remaining pool, or to a subset thereof, with amplification reaction reagents and the reaction will be incubated at a temperature regimen conducive to amplification. This second permutation-specific amplification will selectively amplify the selected polynucleotides.

Generally, the above amplification embodiments provide a mechanism for the selective amplification of particular polynucleotides present in a pool of polynucleotides such that polynucleotides having characteristics of interest may be targeted for further analysis, use, or experimentation. This method may be utilized in a wide variety of molecular biology contexts. For instance, nucleic acid molecules present within any pool of nucleic acid molecules may be synthesized to include one more variable priming sequences. The nucleic acid molecules may be variants of one or more sequences of interest, or may represent a plurality of sequences of interest. The nucleic acid molecules may be amplified and a subset of the amplicons, templates, or a mixture thereof, may be sequenced, as described above. Permutation-specific primers may then be used to amplify selected nucleic acid molecules having one or more particular sequences.

The present invention encompasses all reasonable combinations of the steps described herein, which may be ordered or applied selectively in a plurality of variations. Insofar as the steps of the invention have been described, any process of the invention that may be readily derived from the combination of these steps is encompassed by the present specification.

### EXAMPLES

The below exemplary methods shall not limit the scope of the invention as otherwise disclosed above. The below exemplary methods are illustrations of a subset of the presently invented methods.

### Example 1: Emulsion Gene Synthesis with Base Oligonucleotides

A plurality of polynucleotides are produced by parallel synthesis, e.g., in emulsion or on a microarray chip. Each set of the synthesized tile oligonucleotides comprises a base oligonucleotide and two or more additional tile oligonucleotides. From 3′ to 5′, the base oligonucleotide includes an identifying sequence (e.g., a cBarcode complementary to a barcode attached to a bead), a universal priming site, a first cleavage site, a hybridization sequence, and a second cleavage site. The cleavage sites are type II restriction enzyme cleavage sites. Each additional, non-base tile oligonucleotide, from 3′ to 5′, includes an identifying sequence (e.g., a cBarcode complementary to a barcode attached to a bead), a universal priming site distinct from that of the base oligonucleotide template, a hybridization segment, and a second, distinct cleavage site. The base oligonucleotide of each of the to-be-synthesized products can be synthesized in multiple spots relative to other tile oligonucleotides, such that the base oligonucleotide will be in molar excess to the other tile oligonucleotides for any single to-be-synthesized product. The base oligonucleotides can be the only oligonucleotides having a 5′ terminal phosphate and may further include a separate universal primer unique to all base oligonucleotides. All other tile oligonucleotides can include a hydroxyl moiety at the 5′ end. Each tile oligonucleotide can include an encoding sequence up to, for example, 100 nucleotides in length. Oligonucleotides can further include a whole or partial RNA polymerase binding site (e.g., a T7 or T3 RNA polymerase binding site). Self-priming can be achieved, for example, by use of an unnatural dNTP base-pair (e.g., a spacer or non-A/C/G/T nucleotide) next to the identifying sequence to stop synthesis before the identifying sequence, with a means for removing the barcode and non-native nucleotide sequence.

Primers corresponding to each priming site are hybridized to the template oligonucleotides and extended using a DNA polymerase. The primers bind 5′ of the 3′ terminus of the template oligonucleotides, such that each tile oligonucleotide has a 3′ overhang. The identifying sequence of each tile oligonucleotide is present in this overhang. The base oligonucleotide primer, but not primers corresponding to non-base tile oligonucleotides, includes a 5′ phosphate, such that the newly synthesized strand of the base oligonucleotide includes a 5′ phosphate. If the synthesis is performed in an emulsion, microarray-synthesized DNA can be chemically-cleaved from the array. A universal primer, thermal-stable DNA polymerase, and dNTPs can be added to the cleaved synthesized DNA. The mix can then be emulsified and the temperature raised to 70° C. to initiate second-strand synthesis. The emulsion can then be broken and the dsDNA collected, e.g., using a hydroxyapatite column.

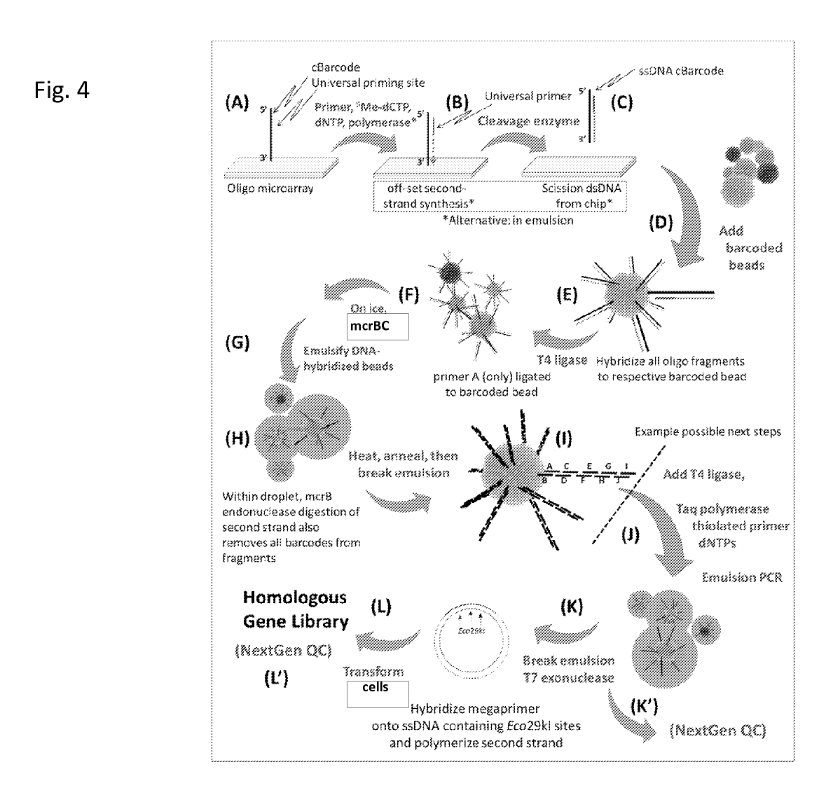

A cleavage agent (e.g., a dsDNA-specific restriction enzyme such as Dpn1) can be used to cleave the 5′ cleavage site (identified as the second cleavage site), liberating the tile oligonucleotides from the chip. The tile oligonucleotides are next contacted with a support having corresponding capture oligonucleotides, to which the tile oligonucleotides hybridize, forming capture complexes. The support can be a bead (e.g., a barcoded Luminex bead). Barcodes complementary to cBarcodes can be, for example, covalently attached to a bead. Such barcodes can be based on, e.g., SNP genotyping primers. Multiple tile oligonucleotides that will be used to compose any single to-be-synthesized product (e.g., a gene) may have the same cBarcode, and will thus hybridize to the same bead. Beads corresponding to particular to-be-synthesized products or to particular polynucleotides can be detectably labeled with correspondingly distinct dyes. A given bead can be attached to multiple copies of a single barcode, or to multiple distinct barcodes and copies thereof. For example, bead-distal and bead-proximal oligonucleotides may recognize distinct barcodes on a bead. The capture complexes are washed to remove unbound primer and the addition of ligase results in covalent attachment of the base oligonucleotide to a corresponding capture oligonucleotide (FIG. 4A-F).

The beads are then emulsified (e.g., in a perfluorocarbon oil) with a cleavage agent capable of cleaving the remaining cleavage site located between the identifying sequence and the hybridization segment (identified as a first cleavage site). Incubation at 95° C. denatures the tile oligonucleotides. An annealing step then allows overlapping hybridization segments to produce a nicked polynucleotide. Hybridization can be accelerated, for example, by using recA protein, recA peptide, or a crowding agent (e.g., polyethylene glycol or hexamine cobalt chloride). A thermal stable DNA polymerase and emulsion PCR can be optionally used to amplify the DNA sequences of the genes on the beads.

The emulsion is then broken and ligase is added to seal the nicks. The polynucleotides remain attached to the beads by the base oligonucleotide. The beads are then sorted by flow cytometry according to the detectable dyes present on each bead. Sorted beads are distributed to microtiter wells, and a PCR reaction is used to amplify the sorted polynucleotide products.

### Example 2: Emulsion Gene Synthesis with Base Oligonucleotides and Selective Degradation of Newly Synthesized Tile Oligonucleotide Strands

A plurality of polynucleotides are produced by parallel synthesis, e.g., in emulsion or on a microarray chip. Each set of tile oligonucleotides comprises a base oligonucleotide and two or more additional tile oligonucleotides. From 3′ to 5′, the base oligonucleotide includes an identifying sequence (e.g., a cBarcode complementary to a barcode attached to a bead), a universal priming site, a first cleavage site, a hybridization sequence, and a second cleavage site. The cleavage sites are type II restriction enzyme cleavage sites. Each additional, non-base tile oligonucleotide, from 3′ to 5′, includes an identifying sequence (e.g., a cBarcode complementary to a barcode attached to a bead), a universal priming site distinct from that of the base oligonucleotide template, a hybridization segment, and a second, distinct cleavage site. The base oligonucleotide of each of the to-be-synthesized products can be synthesized in multiple spots relative to other tile oligonucleotides, such that the base oligonucleotide will be in molar excess to the other tile oligonucleotides for any single to-be-synthesized product. The base oligonucleotides can be the only oligonucleotides having a 5′ terminal phosphate and may further include a separate universal primer unique to all base oligonucleotides. All other tile oligonucleotides can include a hydroxyl moiety at the 5′ end. Each tile oligonucleotide can include an encoding sequence up to, for example, 100 nucleotides in length. Oligonucleotides can further include a whole or partial RNA polymerase binding site (e.g., a T7 or T3 RNA polymerase binding site). Self-priming can be achieved, for example, by use of an unnatural dNTP base-pair (e.g., a spacer or non-A/C/G/T nucleotide) next to the identifying sequence to stop synthesis before the identifying sequence, with a means for removing the barcode and non-native nucleotide sequence.

Primers corresponding to each priming site are hybridized to the template oligonucleotides and extended using a DNA polymerase (FIG. 4A-C). The primers bind 5′ of the 3′ terminus of the template oligonucleotides, such that each tile oligonucleotide has a 3′ overhang. The identifying sequence of each tile oligonucleotide is present in this overhang. The base oligonucleotide primer, but not primers corresponding to non-base tile oligonucleotides, includes a 5′ phosphate, such that the newly synthesized strand of the base oligonucleotide includes a 5′ phosphate. In addition, the base oligonucleotide primer, but not primers corresponding to non-base tile oligonucleotides, includes four 5′ phosphorothioates, such that the newly synthesized strand of each base oligonucleotide is a protected strand. The primers corresponding to non-base tile oligonucleotides do not have four 5′ phosphorothioates, such that the newly synthesized strand of each of these tile oligonucleotides is a non-protected strand. If the synthesis is performed in an emulsion, microarray-synthesized DNA can be chemically-cleaved from the array. A universal primer, thermal-stable DNA polymerase, and dNTPs can be added to the cleaved synthesized DNA. The mix can then be emulsified and the temperature raised to 70° C. to initiate second-strand synthesis. The emulsion can then be broken and the dsDNA collected, e.g., using a hydroxyapatite column.

Next, a cleavage agent (e.g., a dsDNA-specific restriction enzyme such as Dpn1) can be used to cleave the 5′ cleavage site (identified as the second cleavage site), liberating the tile oligonucleotides from the chip. The tile oligonucleotides are next contacted with a support having corresponding capture oligonucleotides, to which the tile oligonucleotides hybridize, forming capture complexes. The support can be a bead (e.g., a barcoded Luminex bead). Barcodes complementary to cBarcodes can be, for example, covalently attached to a bead. Such barcodes can be based on, e.g., SNP genotyping primers. Multiple tile oligonucleotides that will be used to compose any single to-be-synthesized product (e.g., a gene) may have the same cBarcode, and will thus hybridize to the same bead. Beads corresponding to particular to-be-synthesized products or to particular polynucleotides can be detectably labeled with correspondingly distinct dyes. A given bead can be attached to multiple copies of a single barcode, or to multiple distinct barcodes and copies thereof. For example, bead-distal and bead-proximal oligonucleotides may recognize distinct barcodes on a bead. The capture complexes are washed to remove unbound primer. Ligase is provided to covalently attach the base oligonucleotides to corresponding capture oligonucleotides (FIG. 4D-E), and the 5Me-dCTP-specific endonuclease mcrBC is added to selectively degrade the newly synthesized tile oligonucleotide strands, excluding the newly synthesized base oligonucleotide strand (FIG. 4F-G). The emulsion is incubated for a sufficient time at 30° C. as to allow the mcrBC endonuclease to digest the synthesized, unprotected strands of DNAs, also resulting in the removal of the extraneous barcode sequences that were added to each fragment. As an alternative method for strand-specific degradation, deoxyuracil can be incorporated, for example, into one strand of a synthesized oligonucleotide and the dut-ung enzymes can be added prior to emulsification. Further alternative enzymes include, for example, type IIs nickase and modified-DNA recognizing enzymes (e.g., DpnI).

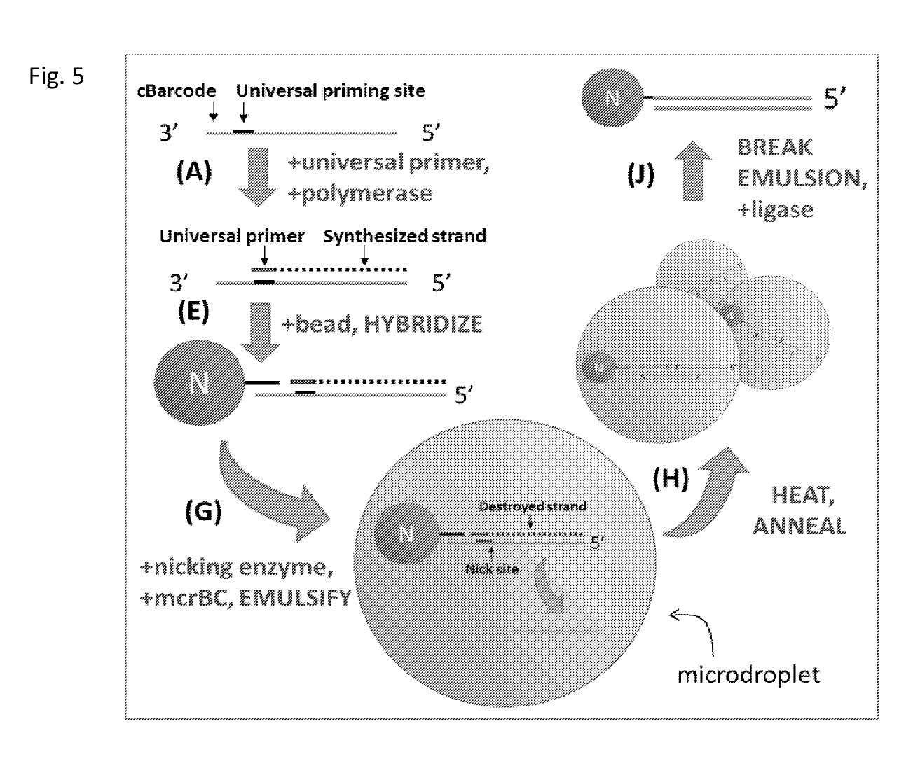

The beads are then emulsified (e.g., in a perfluorocarbon oil) with a cleavage agent capable of cleaving the remaining cleavage site located between the identifying sequence and the hybridization segment (identified as a first cleavage site). Incubation at 95° C. denatures the tile oligonucleotides. An annealing step then allows overlapping hybridization segments to produce a nicked polynucleotide. Hybridization can be accelerated, for example, by using recA protein, recA peptide, or a crowding agent (e.g., polyethylene glycol or hexamine cobalt chloride). The emulsion is then broken and ligase is added to seal the nicks (FIG. 4H-J). The polynucleotides remain attached to the beads by the base oligonucleotide (FIG. 5). A thermal stable DNA polymerase and emulsion PCR can be used to amplify the DNA sequences of the genes on the beads. The beads are then sorted by flow cytometry according to the detectable dyes present on each bead. Sorted beads can be distributed to microtiter wells, and a PCR reaction is used to amplify the sorted polynucleotide products. The polynucleotide products can also be cloned into suitable vectors to form a library (e.g., a homologous gene library) and/or undergo next generation sequencing for quality control (FIG. 4K-L). Passing synthesis quality control can be defined as an ability to isolate at least 1 error-free sequence for >95% of all 1000-nucleotide variant gene segments.

### Example 3: Emulsion Gene Synthesis with Base Oligonucleotides and Selective Degradation of Newly Synthesized Tile Oligonucleotide Strands within an Emulsion

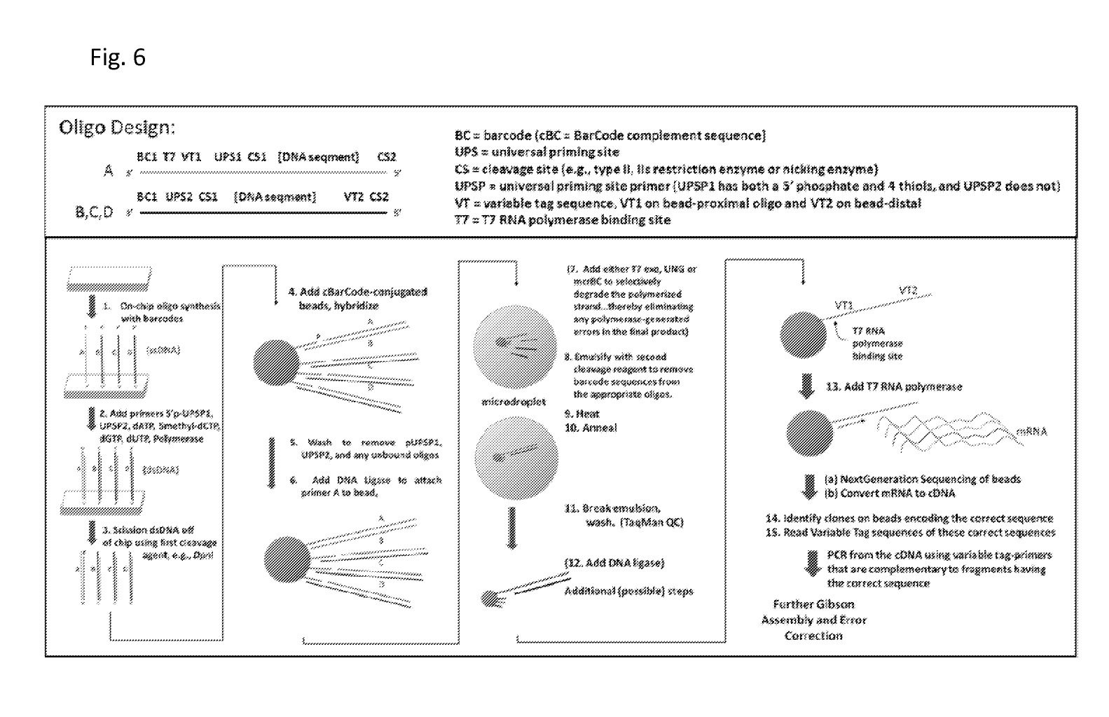

A plurality of polynucleotides are produced by parallel synthesis in emulsion. Each set of tile oligonucleotides comprises a base oligonucleotide and two or more additional tile oligonucleotides. From 3′ to 5′, the base oligonucleotide includes an identifying sequence, a universal priming site, a first cleavage site, a hybridization sequence, and a second cleavage site. The cleavage sites are type II restriction enzyme cleavage sites. Each additional, non-base tile oligonucleotide, from 3′ to 5′, includes an identifying sequence, a universal priming site distinct from that of the base oligonucleotide template, a hybridization segment, and a second, distinct cleavage site (FIG. 6, top panel). Primers corresponding to each priming site are hybridized to the template oligonucleotides and extended. The primers bind 5′ of the 3′ terminus of the template oligonucleotides, such that each tile oligonucleotide has a 3′ overhang. The identifying sequence of each tile oligonucleotide is present in this overhang. The base oligonucleotide primer, but not primers corresponding to non-base tile oligonucleotides, includes a 5′ phosphate, such that the newly synthesized strand of the base oligonucleotide includes a 5′ phosphate. In addition, the base oligonucleotide primer, but not primers corresponding to non-base tile oligonucleotides, includes four 5′ phosphorothioates, such that the newly synthesized strand of each base oligonucleotide is a protected strand. The primers corresponding to non-base tile oligonucleotides do not have four 5′ phosphorothioates, such that the newly synthesized strand of each of these tile oligonucleotides is a non-protected strand. Further, the base oligonucleotide template includes, from 3′ to 5′, a T7 RNA polymerase binding site and a variable tag sequence between the identifying sequence and the priming site. The other tile oligonucleotide templates include a variable tag sequence distinct from that of the base oligonucleotide template between the hybridization sequence and second cleavage site.

Next, a cleavage agent, DpnI, is used to cleave the 5′ cleavage site (identified as the second cleavage site), liberating the tile oligonucleotides from the chip. The tile oligonucleotides are next contacted with a support having corresponding capture oligonucleotides, to which the tile oligonucleotides hybridize, forming capture complexes. The support is a bead. Beads corresponding to distinct polynucleotides are detectably labeled with correspondingly distinct dyes. The capture complexes are washed to remove unbound primer and resuspended in T4 ligase buffer with T4 DNA Ligase. Ligase is provided to covalently attach the base oligonucleotides to corresponding capture oligonucleotides. The mixture is incubated at 37° C. for a period sufficient for the enzyme to establish a covalent bond between at least one base oligonucleotide having a 5′ phosphate and a corresponding capture oligonucleotide.

The capture complex is then emulsified with a cleavage agent capable of cleaving the remaining cleavage site located between the identifying sequence and the hybridization segment (identified as a first cleavage site). The emulsification further includes an enzyme capable of selectively degrading one strand of each tile oligonucleotide except the base oligonucleotide. The enzyme may be T7 exonuclease, UNG, or mcrBC. The emulsion may then be heated and annealed to produce one or more polynucleotides. Subsequently, the emulsion may be broken, the beads washed, and the produced polynucleotides treated with ligase. In subsequent step, the bead-attached polynucleotides are contacted with T7 RNA polymerase to produce mRNA. The bead-attached polynucleotides are sequenced by next generation sequencing. From the resultant sequence data, clones with correct sequences are identified and the variable tag sequences are read. Further, the produced mRNA is converted to cDNA, and a PCR reaction is performed on the cDNA using primers complementary to the variable tags may be used in a further Gibson Assembly reaction and in a method of error correction (FIG. 6).

### Example 4: Emulsion Gene Synthesis with Base Oligonucleotides and Selective Degradation of Newly Synthesized Tile Oligonucleotide Strands within an Emulsion

Several thousand template oligonucleotides, each having an identifying sequence, are synthesized on an Oligo Library Synthesis (OLS) chip under conditions of reduced depurination. A complementary strand is polymerized onto the chip-synthesized template. The double-stranded products are liberated from the chip by cleavage at a cleavage site and pooled in bulk, producing tile oligonucleotides. The cleavage reagent that cleaves the cleavage site is a nicking, type Ils restriction enzyme, or DpnI. The tile oligonucleotides have identifying sequences. Alternatively, second strand synthesis may be performed in an emulsion following cleavage of the single-stranded oligonucleotides from the chip (FIG. 4). The emulsion may include the cleaved synthesized oligonucleotides, primers corresponding to the universal priming sites, and DNA polymerase. The emulsion is incubated at a temperature regimen that allows second strand synthesis and then broken to pool the double-stranded products in bulk, producing tile oligonucleotides.

A mobile solid support (a bead) having capture oligonucleotides corresponding to one or more of the tile oligonucleotides is contacted with the tile oligonucleotides. The beads are hybridized with the bulk solution and then washed to remove unbound tile oligonucleotides. The beads are then emulsified with one or more cleavage agents and SO-PCR reaction reagents to produce a water-in-oil emulsion. Within the emulsion, the cleavage agents cleave the tile oligonucleotides between the hybridization sequence and the identifying sequence.

UNG, T7 exonuclease or mcrBC are used to selectively destroy one strand of the dsDNA in the emulsion. The emulsion is then incubated with ligase at a temperature regimen sufficient to ligate the base oligonucleotide to the corresponding capture oligonucleotide. The emulsion is then broken and the beads are washed. Beads are subsequently re-emulsified with PCR reagents and incubated at a temperature regimen effective to amplify the polynucleotide. Amplicons or polynucleotides may be sequenced with a Next Generation sequencing instrument. Subsequent amplification with primers specific to a sequenced variable tag may be used to selectively amplify particular polynucleotides. The polynucleotides generated by emulsion gene synthesis may be used in further cloning steps, such as those described, for example, in PCT Publication No. WO 2014/134166, which is incorporated by reference herein in its entirety.

### Example 5: Orthogonal PCR of Variable-Tagged Sequences to Recover Specific Clones Before or after DNA Sequencing

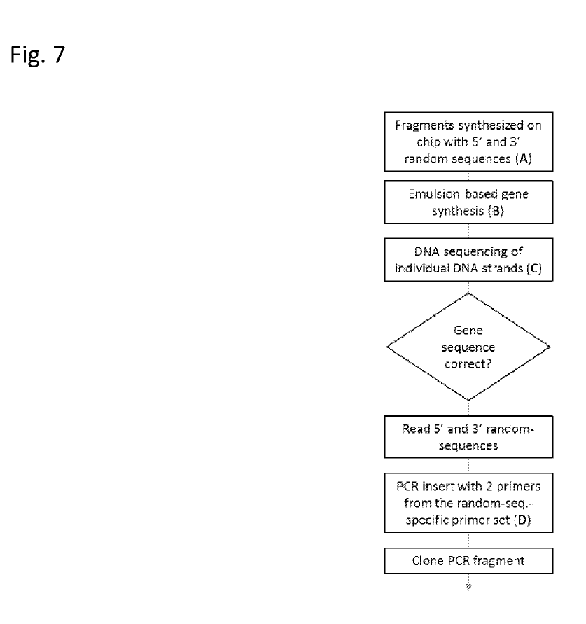

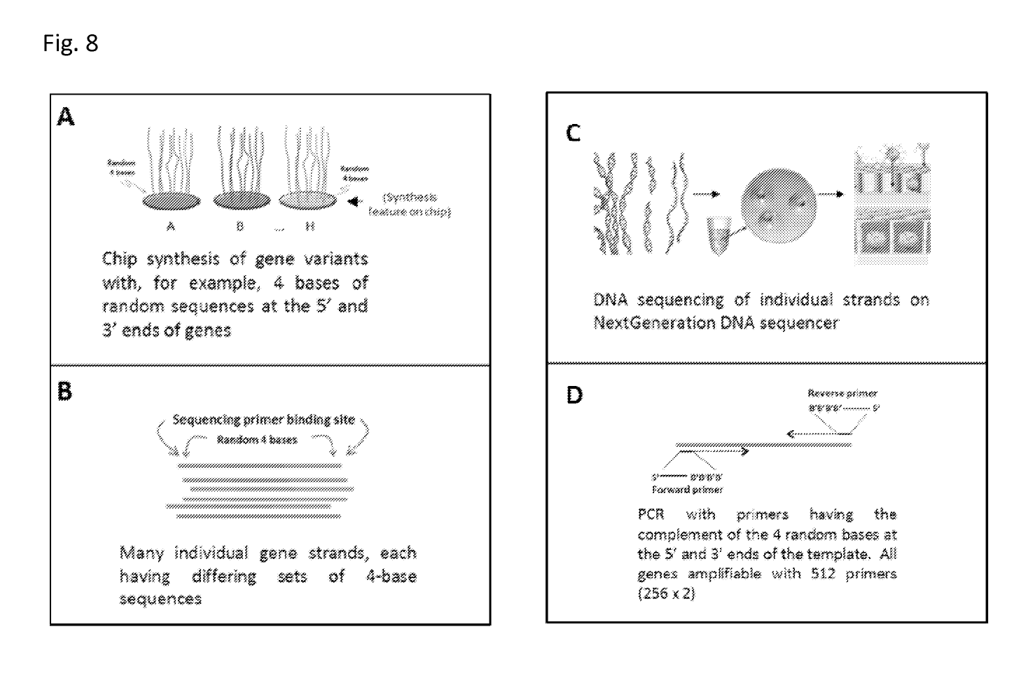

Tile oligonucleotides corresponding to a polynucleotide are synthesized to include a random 4-nt variable tag sequences near both termini of the polynucleotide. The set includes a base oligonucleotide such that after adjoining extension of the set within an emulsion of the present invention, the polynucleotide remains attached to the support, a bead. The bead is treated with T7 RNA polymerase, resulting in T7 RNA polymerase run off from an RNA polymerase binding site on one end of the bead-attached polynucleotide. RNA transcripts are especially useful because they consist of many copies of a single strand. RNA transcription is followed by long-range and/or paired-end next generation sequencing of the polynucleotide that is still attached to the beads in order to identify both the specific eight bases of random sequence on each polynucleotide and to identify polynucleotides having the correct sequence. Specifically selected polynucleotides present in the pool of RNA transcripts are amplified using primers selected based on the variable tags of the selected sequence. Selected amplified polynucleotides are subsequently cloned (see, e.g., FIGS. 6, 7, and 8). This method theoretically permits amplification of a single sequence from 65,536 (=4⁸) distinct sequences using orthogonal PCR. Larger random sequences or the use of unnatural nucleotides to extend the radix of DNA-encoding beyond base₄ are used if extra encoding is needed. For instance, if employing a third set of base-pairs (i.e., other than A=T or G=T), a four-nucleotide variable region enables a base₆ radix and (6⁴×6⁴=6⁸=) 1,679,616 possible random sequences using just (2×6⁴=) 2,592 different orthogonal PCR primers. This results in a five-fold increase of coding efficiency [i.e., a 25-fold increase in coding capacity with a 5-fold increase in the number of PCR primers needed=(6⁸/2,592)/(4⁸/512)=5.0625].

### Example 6: Synthesis of Variant Complementarity Determining Regions

Methods of the invention can involve, for example, systematically generating all of the possible permutations of 20 amino acids at every residue of a protein. For example, a protein of 301 amino acids would require the synthesis of 300 (assuming the first amino acid would always be methionine)×20 changes per residue=6,000 possible genes. Although this method can be applied to nearly any protein, in one particular example, it may be used to model somatic hyper-mutagenesis of the complementarity determining regions (CDRs) of an antibody (e.g., CDR-L1, CDR-L2, CDR-L3, CDR-H1, CDR-H2, and/or CDR-H3), and candidates screened, for example, by phage display.

### OTHER EMBODIMENTS

While the invention has been described in connection with the specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

1. A method of generating a polynucleotide, said method comprising the steps of:
(a) providing a support and two or more tile oligonucleotides, wherein said two or more tile oligonucleotides comprise overlapping, complementary segments of said polynucleotide, and said support has one or more capture oligonucleotides, wherein a segment of each of said tile oligonucleotides is complementary to at least one of said capture oligonucleotides;
(b) contacting said support with said tile oligonucleotides, wherein said tile oligonucleotides hybridize to said capture oligonucleotides, thereby forming a capture complex;
(c) emulsifying said capture complex in an emulsion medium, said emulsion medium further comprising reaction reagents sufficient to carry out an adjoining extension reaction, wherein said emulsion medium forms an emulsion droplet comprising said capture complex and said reaction reagents; and
(d) incubating said emulsion droplet at a temperature regimen that allows adjoining extension of said two or more tile oligonucleotides, thereby generating said polynucleotide.

2. The method of claim 1, wherein each of said tile oligonucleotides comprises an identifying sequence.

3. The method of claim 2, wherein all of said tile oligonucleotides comprise the same identifying sequence.

4. The method of claim 2, wherein said tile oligonucleotides comprise a plurality of distinct identifying sequences.

5. The method of claim 4, wherein one of said tile oligonucleotides is a base oligonucleotide that comprises a first identifying sequence distinct from that of the remaining tile oligonucleotides.

6. The method of any one of claims 1-5, wherein one or more of said tile oligonucleotides are provided as double-stranded tile oligonucleotides.

7. The method of claim 6, wherein one or more of said double-stranded tile oligonucleotides are prepared from one or more single-stranded template oligonucleotides prior to providing said tile oligonucleotides.

8. A method of generating a polynucleotide, said method comprising the steps of:
(a) synthesizing two or more double-stranded tile oligonucleotides from one or more single-stranded template oligonucleotides by providing one or more primers capable of hybridizing to one or more of said template oligonucleotides at one or more priming sequences, hybridizing said primers to said priming sequences, and extending said primers to produce one or more double-stranded tile oligonucleotides comprising a template strand and a newly synthesized strand;
(b) providing a support and two or more of said tile oligonucleotides, wherein said two or more tile oligonucleotides are overlapping, complementary segments of said polynucleotide, and said support has one or more capture oligonucleotides, wherein a segment of each of said tile oligonucleotides is complementary to at least one of said capture oligonucleotides;
(c) contacting said support with said tile oligonucleotides, wherein said tile oligonucleotides hybridize to said capture oligonucleotides, thereby forming a capture complex, and emulsifying said capture complex in an emulsion medium, said emulsion medium further comprising reaction reagents sufficient to carry out an adjoining extension reaction, wherein said emulsion medium forms an emulsion droplet comprising said capture complex and said reaction reagents; and
(d) incubating said emulsion droplet at a temperature regimen that allows adjoining extension of said two or more tile oligonucleotides, thereby generating said polynucleotide.

9. The method of claim 8, wherein two or more of said template oligonucleotides comprise identical priming sequences.

10. The method of claim 8, wherein said template oligonucleotides comprise a plurality of priming sequences.

11. The method of any one of claims 8-10, wherein said synthesizing is by solid state synthesis from template oligonucleotides affixed at the 5′ terminus to a solid state synthesis structure, said synthesizing step further comprising providing a cleavage reagent after said extending and incubating said solid state synthesis structure with said cleavage reagent, wherein said template oligonucleotides further comprise one or more cleavage sites for said cleavage reagent positioned 5′ of the segment of said template oligonucleotide corresponding to said polynucleotide, thereby producing one or more tile oligonucleotides each comprising a template strand and a newly synthesized strand.

12. The method of any one of claims 8-10, wherein said synthesizing is by solid state synthesis from template oligonucleotides affixed at the 3′ terminus to a solid state synthesis structure, said synthesizing step further comprising providing a cleavage reagent after said extending and incubating said solid state synthesis structure with said cleavage reagent, wherein said template oligonucleotides further comprise one or more cleavage sites for said cleavage reagent positioned 3′ of the segment of said template oligonucleotide corresponding to said polynucleotide, thereby producing one or more tile oligonucleotides each comprising a template strand and a newly synthesized strand.

13. The method of any one of claims 8-10, wherein said template oligonucleotides are initially affixed at the 5′ terminus to a solid state synthesis structure, said synthesizing step further comprising providing a cleavage reagent prior to said extending and incubating said solid state synthesis structure with said cleavage reagent, wherein said template oligonucleotides further comprise one or more cleavage sites for said cleavage reagent positioned 5′ of the segment of said template oligonucleotide corresponding to said polynucleotide, thereby producing one or more free single-stranded template oligonucleotides prior to said extending.

14. The method of any one of claims 8-10, wherein said template oligonucleotides are initially affixed at the 3′ terminus to a solid state synthesis structure, said synthesizing step further comprising providing a cleavage reagent prior to said extending and incubating said solid state synthesis structure with said cleavage reagent, wherein said template oligonucleotides further comprise one or more cleavage sites for said cleavage reagent positioned 3′ of the segment of said template oligonucleotide corresponding to said polynucleotide, thereby producing one or more free single-stranded template oligonucleotides prior to said extending.

15. The method of any one of claims 8-14, wherein said primers capable of hybridizing to one or more of said template oligonucleotides are primers for a strand-displacing polymerase and wherein said extending is by a single round of strand-displacing extension.

16. The method of any one of claims 13-15, wherein said extending step is performed in an emulsion comprising said single-stranded template oligonucleotides, said primers, and said polymerase.

17. The method of claim 16, further comprising the step of breaking said emulsion, thereby producing a solution comprising one or more double-stranded tile oligonucleotides.

18. The method of any one of claims 8-17, wherein each of said template oligonucleotides comprises an identifying sequence, whereby each double-stranded tile oligonucleotide comprises the identifying sequence present in the template oligonucleotide from which it was synthesized.

19. The method of claim 18, wherein said priming sequence is 5′ of said identifying sequence, thereby generating a tile oligonucleotide in which the 3′ end of said template strand comprises a single-stranded 3′ overhang that extends beyond the 5′ end of said newly synthesized strand and comprises said identifying sequence.

20. The method of claim 19, wherein one or more of said primers comprises a 5′ phosphate and hybridizes specifically to a template oligonucleotide encoding a base oligonucleotide, whereby said synthesis results in said base oligonucleotide comprising a newly synthesized strand comprising a 5′ phosphate, and said step of contacting said support to said tile oligonucleotides further comprises contacting ligase to said support and incubating said ligase, support, and tile oligonucleotides together prior to said emulsification, whereby one or more of said newly synthesized strands comprising a 5′ phosphate are covalently joined by the activity of said ligase to a capture oligonucleotide of said capture complex.

21. The method of any one of claims 1-20, wherein one or more of said tile oligonucleotides hybridized to said capture oligonucleotides in said capture complex further comprise cleavage sites positioned such that cleavage at one or more of said cleavage sites liberates from said capture complex a portion of one or more of said tile oligonucleotides comprising the segment corresponding to said polynucleotide, said method further comprising contacting said capture complex with one or more cleavage reagents.

22. The method of claim 21, wherein all of said tile oligonucleotides comprise a cleavage site.

23. The method of claim 21, wherein one or more of said tile oligonucleotides are base oligonucleotides and all of said tile oligonucleotides except said base oligonucleotides comprise a cleavage site.

24. The method of any one of claims 8-23, wherein one strand of one or more of said double-stranded tile oligonucleotides is protected and the second strand is non-protected, said method further comprising the step of selectively degrading said non-protected strand over said protected strand prior to said incubation at a temperature regimen that allows adjoining extension, thereby producing a single-stranded tile oligonucleotide.

25. The method of claim 24, wherein said non-protected strand comprises two or fewer 5′ phosphorothioate groups, said protected strand comprises three or more 5′ phosphorothioate groups, and said degrading comprises incubating said tile oligonucleotides with an enzyme capable of selectively degrading a strand having two or fewer 5′ phosphorothioate groups over a strand having three or more 5′ phosphorothioate groups.

26. The method of claim 25, wherein said enzyme capable of selective degradation is T7 exonuclease or lambda exonuclease.

27. The method of claim 24, wherein said non-protected strand comprises methylated nucleobases, said protected strand lacks methylated nucleobases, and said degrading comprises incubating said tile oligonucleotides with an enzyme capable of selectively degrading a methylated strand over a strand that is not methylated.

28. The method of claim 27 wherein said non-protected strand comprises methylated adenine nucleobases and said enzyme capable of selective degradation is DpnI.

29. The method of claim 27 wherein said non-protected strand comprises methylated cytosine nucleobases and said enzyme capable of selective degradation is mcrBC, or said non-protected strand comprises deoxyuracil and said enzyme capable of selective degradation is dut.

30. The method of claim 24, wherein said non-protected strand lacks methylated nucleobases, said protected strand comprises methylated nucleobases, and said degrading comprises incubating said tile oligonucleotides with an enzyme capable of selectively degrading a non-methylated strand over a methylated strand.

31. The method of claim 30 wherein said protected strand comprises methylated cytosine or guanine nucleobases and said enzyme that selectively degrades non-methylated nucleic acids is Sau3AI.

32. The method of claim 24, wherein said non-protected strand comprises uracil, said protected strand lacks uracil, and said degrading comprises incubating said tile oligonucleotides with an enzyme capable of selectively degrading a uracilated strand over a strand that is not uracilated, thereby producing a single-stranded tile oligonucleotide.

33. The method of claim 32 wherein said enzyme that selectively degrades uracilated nucleic acids is a uracil-DNA glycosylase.

34. The method of any one of claims 24-33, wherein said protected strand is said template strand.

35. The method of claim 34, wherein said step of selectively degrading said template strand occurs after said contacting of said capture complex with said cleavage reagent.

36. The method of any one of claims 24-33, wherein said protected strand is said newly synthesized strand.

37. The method of claim 35, wherein said step of selectively degrading said newly synthesized strand occurs prior to said contacting of said capture complex with said cleavage reagent.

38. The method of claim 35, wherein said step of selectively degrading said newly synthesized strand occurs prior to said emulsifying.

39. The method of claim 35, wherein said step of selectively degrading said newly synthesized strand occurs after said contacting of said capture complex with said cleavage reagent.

40. The method of any one of claims 1-39, wherein each of said tile oligonucleotides is 20 bp to 2 kb in length.

41. The method of any one of claims 1-40, wherein said support comprises capture nucleotides synthesized to hybridize to the identifying sequences of tile oligonucleotides corresponding to a single polynucleotide.

42. The method of any one of claims 1-40, wherein said support comprises capture nucleotides synthesized to hybridize to the identifying sequences of tile oligonucleotides corresponding to two to ten distinct polynucleotides.

43. The method of any one of claims 1-42, wherein said support comprises 1 to 1,000 distinct capture oligonucleotides, preferably 2 to 50 distinct capture oligonucleotides.

44. The method of any one of claims 1-43, wherein said reaction reagents are sufficient to carry out a SO-PCR reaction and said adjoining extension reaction comprises SO-PCR.

45. The method of any one of claims 1-43, wherein said reaction reagents are sufficient to carry out a Gibson Assembly reaction and wherein said adjoining extension reaction comprises a Gibson Assembly reaction.

46. The method of any one of claims 1-43, wherein said extension reaction comprises a temperature regimen sufficient to denature and subsequently reanneal overlapping, complementary sequences of said tile oligonucleotides.

47. The method of any one of claims 1-46, wherein said emulsifying comprises a plurality of supports and the resulting emulsion comprises a plurality of droplets.

48. The method of claim 47, wherein each droplet of said emulsion contains 0-10 supports.

49. The method of claim 48, wherein each droplet of said emulsion contains, on average, 0-2 supports.

50. The method of claim 49, wherein each droplet of said emulsion contains, on average, 1 support.

51. The method of any one of claims 1-50, further comprising the step of
(e) breaking said emulsion, thereby producing a solution comprising one or more supports and one or more polynucleotides.

52. The method of claim 51, further comprising the step of purifying said polynucleotides.

53. The method of claim 51, further comprising the step of purifying said supports.

54. The method of claim 53, wherein said supports comprise one or more detectable labels, and said method further comprises, after said step of breaking said emulsion, the step of sorting said supports according to said one or more detectable labels.

55. The method of any one of claims 51-54, further comprising incubating said polynucleotides with ligase after breaking said emulsion, thereby forming covalent bonds at nicks in said polynucleotide.

56. The method of any one of claims 1-55, wherein said polynucleotide is 50 bp-20 kb in length, preferably 100 bp-10 kb.

57. The method of any one of claims 47-56, said method further comprising generating a plurality of distinct polynucleotides, wherein said emulsion comprises a plurality of supports corresponding to distinct polynucleotides, such that said incubation of said emulsion at a temperature regimen that allows adjoining extension results in the generation of a plurality of distinct polynucleotides, each polynucleotide being generated within an emulsion droplet comprising the corresponding support.

58. The method of any one of claims 51-57, further comprising the step of amplifying said polynucleotides.

59. The method of any one of claims 51-57, wherein two or more polynucleotides present in an emulsion comprise one or more variable priming sequences having one or more distinct permutations positioned 3′ of a sequence of interest on one or both strands of each of said polynucleotides, said method further comprising the steps of:
(f) providing one or more permutation-specific primers and amplification reaction reagents;
(g) contacting said polynucleotides with said permutation-specific primers in the presence of said amplification reaction reagents; and
(h) incubating said polynucleotides together with said permutation-specific primers in the presence of said amplification reaction reagents at a temperature regimen that allows amplification, thereby selectively amplifying polynucleotides having a particular permutation of a variable priming sequence.

60. The method of any one of claims 51-57, wherein two or more polynucleotides present in an emulsion comprise one or more variable priming sequences having one or more distinct permutations and one or more non-variable priming sequences positioned 3′ of a sequence of interest on one or both strands of said polynucleotide, said method further comprising the steps of:
(f) providing one or more primers capable of hybridizing to said non-variable priming sequences and a first set of amplification reaction reagents;
(g) contacting said polynucleotides to said non-variable priming sequence primers in the presence of said first set of amplification reaction reagents;
(h) incubating said polynucleotides together with said non-variable priming sequence primers and said first set of amplification reaction reagents at a temperature regimen that allows amplification, thereby producing amplicons of polynucleotides having said non-variable priming sequence;
(i) removing and sequencing a subset of amplicons to identify polynucleotide sequences having a particular sequence and one or more associated variable priming sequence permutations;
(j) providing one or more permutation-specific primers and a second set of amplification reaction reagents;
(k) contacting said remaining polynucleotides and/or amplicons with said permutation-specific primers in the presence of said second set of amplification reaction reagents; and
(l) incubating said remaining polynucleotides and/or amplicons together with said permutation-specific primers in the presence of said amplification reaction reagents at a temperature regimen that allows amplification, thereby selectively amplifying polynucleotide sequences having a particular permutation of a variable priming sequence.

61. The method of any one of claims 1-60, wherein said support is a bead, chip, tube, or well.

62. The method of claim 61, wherein said bead is a magnetic bead.

63. The method of claim 61 or 62, wherein said bead is labeled.

64. The method of claim 63, wherein said label is a colored label or a fluorescent label.

65. The method of claim 64, wherein said fluorescent label is a dye or fluorescent protein.

66. The method of any one of claims 1-65, wherein said emulsifying step comprises emulsifying said capture complex in a water-in-oil emulsion.

67. The method of claim 66, wherein said water-in-oil emulsion is a water-in-perfluorocarbon oil emulsion.

68. The method of any one of claims 1-67, wherein said emulsifying step comprises the use of a mechanical device to emulsify said capture complex in said emulsion medium.

69. The method of claim 68, wherein said mechanical device is a stirrer, homogenizer, colloid mill, ultrasound, membrane emulsification device, or vortex.

70. The method of any one of claims 1-69, wherein said emulsion medium further comprises a recA protein, recA peptide, or a crowding agent.

71. The method of claim 70, wherein said crowding agent is polyethylene glycol or hexamine cobalt chloride.

72. The method of any one of claims 1-71, wherein said polynucleotide encodes one or more complementarity determining regions.

73. The method of claim 72, wherein said polynucleotide encodes CDR-L1, CDR-L2, CDR-L3, CDR-H1, CDR-H2, and/or CDR-H3.

74. A method for the selective amplification of one or more synthesized polynucleotides comprising the steps of:
(a) providing a pool of polynucleotides, each polynucleotide comprising one or more variable priming sequences on one or both strands of said polynucleotide 3′ of a sequence to be amplified, one or more permutation-specific primers, and amplification reaction reagents, wherein said polynucleotides present in said pool comprise one or more distinct permutations of one or more of said variable priming sequences;
(b) contacting said pool of synthesized polynucleotides with said permutation-specific primers in the presence of said amplification reaction reagents; and
(c) incubating said polynucleotides together with said permutation-specific primers in the presence of said amplification reaction reagents at a temperature regimen that allows amplification, thereby selectively amplifying polynucleotides having a particular distinct permutation of a variable priming sequence.

75. The method of any one of claims 51 to 74, wherein two or more of said polynucleotides comprise distinct permutations of one or more of said variable priming sequences

76. The method of any one of claims 51 to 75, wherein said variable priming sequence comprises two or more variable nucleotide positions.

77. The method of claim 75, wherein said variable priming sequence comprises two to six variable nucleotide positions.

78. The method of claim 77, wherein said variable priming sequence comprises four variable nucleotide positions.

79. The method of any of claims 74 to 78, wherein said variable priming sequence consists of eight to thirty nucleotides.

80. The method of any of claims 74 to 79, wherein the non-variable nucleotide positions are constant positions.

81. The method of any of claims 74 to 80, wherein two or more polynucleotides that are otherwise identical comprise distinct variable priming sequence permutations.

82. The method of claim 81, wherein one or more of said two or more polynucleotides that are otherwise identical encodes a variant of the sequence of interest.

83. The method of claim 82, wherein said permutation-specific primers hybridize selectively to the permutation present in a polynucleotide encoding the sequence of interest.

84. The method of claim 82, wherein said permutation-specific primers hybridize selectively to the permutation present in a polynucleotide encoding a variant of said sequence of interest.

85. The method of any of claims 74 to 84, wherein said variable nucleotide position comprises an adenine, guanine, cytosine, thymine, or uracil nucleotide.

86. The method of any one of claims 74 to 85, wherein said variable nucleotide position comprises a nucleotide other than adenine, guanine, cytosine, thymine, or uracil.

87. The method of claim 86, wherein said variable nucleotide position comprises a synthetic nucleotide.

88. The method of any one of claims 74 to 87, wherein the variable nucleotide positions are contiguous.

89. The method of any one of claims 74 to 88, wherein the variable nucleotide positions are not contiguous.

90. The method of any one of claims 74 to 89, wherein an amplicon of a polynucleotide having a distinct variable priming site permutation is sequenced and said polynucleotide is consequently selected for said selective amplification.

91. A complex comprising a support and one or more tile oligonucleotides, wherein said support comprises one or more capture oligonucleotides hybridized to said one or more tile oligonucleotides, wherein said tile oligonucleotides are complementary, overlapping segments of a polynucleotide.

92. A solution comprising two or more tile oligonucleotides and one or more supports, wherein said two or more tile oligonucleotides are complementary, overlapping segments of a polynucleotide and said supports comprise two or more capture oligonucleotides capable of hybridizing to said tile oligonucleotides.

93. A method of performing an adjoining extension reaction in an emulsion, wherein an emulsion droplet comprising segments comprising sequences to be adjoined and reaction reagents sufficient to carry out an adjoining extension reaction are incubated at a temperature regimen sufficient to perform said adjoining extension reaction, whereby said sequences to be adjoined produce a single polynucleotide.

94. The method of claim 93, wherein said reaction reagents are sufficient to carry out a SO-PCR reaction and wherein said adjoining extension comprises SO-PCR.

95. The method of claim 93, wherein said reaction reagents are sufficient to carry out a Gibson Assembly reaction and wherein said adjoining extension comprises a Gibson Assembly reaction.
